(19) United States
(12) Patent Application Publication
(10) Pub. No.: US 2023/0203111 A1
(43) Pub. Date: Jun. 29, 2023

(54) Ferritin Heavy Chain Subunit-Based Conjugates and Application Thereof

(71) Applicant: KUNSHAN XINYUNDA BIOTECH CO., LTD., Kunshan, Jiangsu (CN)

(72) Inventors: Tianyi Ke, Kunshan, Jiangsu (CN); Hui Ding, Kunshan, Jiangsu (CN); Dehui Yao, Kunshan, Jiangsu (CN); Fang Lao, Kunshan, Jiangsu (CN); Haiyong Yu, Kunshan, Jiangsu (CN); Jianwei Cheng, Kunshan, Jiangsu (CN); Fangxing Ouyang, Kunshan, Jiangsu (CN)

(21) Appl. No.: 17/926,542

(22) PCT Filed: May 17, 2021
(86) PCT No.: PCT/CN2021/094089

§ 371 Date: Nov. 18, 2022

(30) Foreign Application Priority Data
May 19, 2020 (CN) 202010424329.6

(51) Int. Cl. C07K 14/47; A61K 38/17; A61K 47/65

(52) U.S. Cl. CPC C07K 14/47; A61K 38/17; A61K 47/65

(57) ABSTRACT

The present invention relates to the field of biological medicines. Specifically, the present invention relates to a conjugate based on a ferritin heavy chain subunit and use thereof.

### TECHNICAL FIELD

The present invention relates to the field of biological medicines. Specifically, the present invention relates to a conjugate based on ferritin heavy chain subunit and use thereof.

### BACKGROUND ART

A ferritin isolated from a human body or other mammal animals is often composed of two different ferritin subunits (H subunit and L subunit), and the molecular weights of the H subunit and the L subunit are respectively 21 KDa and 19 KDa. A typical ferritin structure is a spherical shell-shaped structure formed by self-assembling 24 light chain/heavy chain subunits, which has an outer diameter of 12 nm and an inner cavity structure with a diameter of 8 nm formed inside.

The single subunit of the ferritin is folded from N terminal into four long α helices and one short α helix and ends at C terminal. After the ferritin is assembled into a complete protein shell, the N terminal of each ferritin subunit is exposed on the outer surface of the protein shell, and the C terminal is folded onto the inner surface of the protein shell. The N terminals of three adjacent ferritin subunits form a triple symmetry axis of ferritin, and the cyclic regions of the flexible amino acids between the fourth α helixes and the fifth α helixes of four ferritin subunits form a quadruple symmetry axis of ferritin.

In 2010, Seaman's laboratory confirmed through cDNA library screening and cell line expression methods that the human H subunit ferritin can specifically bind to human somatic cell membrane receptor TfR1 (transferrin receptor 1), and the human L subunit ferritin has no such the binding function.

The human H ferritin (HFn) can target tumor cells through TfR1, however, in view of the heterogeneity and complexity of tumors, targeting for a single target is usually difficult to meet clinical tumor diagnosis and treatment requirements. To solve this problem, functional proteins (such as antibodies, ligand peptides that can bind to receptors, small molecule peptide drugs, apoptosis propeptides and fluorescent proteins) are expressed at the N-terminal or C-terminal of HFn through fusion protein expression to confer HFn with targeting, traceability or therapeutic (literatures for construction of a fusion mode can be seen in WO2017039382A1, WO2016122259A1, KR-2018008349, WO2013055058A2, WO2018012952A1, CN104017088A, “Ferritin nanogels with biologically orthogonal alignment for vacuum targeting and imaging”, etc.).

The construction method of fusion proteins is a biological construction method, which has the disadvantages of complex method steps, large organism influence, low efficiency, long period, high cost and high failure rate. Design of each ferritin-loaded drug needs to undergo a whole process including gene sequence design, protein expression and purification and impurity control, which is difficult to meet the needs of high-throughput screening and determination of ferritin conjugated drugs, and is not conducive to the medicine development of ferritin drugs. In addition, the fusion construction manner inevitably changes the primary amino acid sequence of the H subunit in HFn. Therefore, after the fusion protein is expressed, it is more expressed as inclusion bodies, or even is not expressed. However, it is also quite uncertain that renaturation of the inclusion body obtains a space structure that has been correctly folded and can be polymerized to form the ferritin spherical body.

### BRIEF DESCRIPTION OF THE DRAWINGS

FIG. 1 shows sodium dodecyl sulfate-polyacrylamide gel electrophoresis (SDS-PAGE) results of 13 mutants.

FIG. 2 shows transmission electron microscopy (TEM) results of HFn mutants.

FIG. 3 shows binding affinity results of unconjugated mutants and the Tfr1 receptor.

FIG. 4 shows conjugation reactivity comparison between cysteine at position 90 and cysteine at position 102.

### SUMMARY OF THE INVENTION

### I. Definition

In the present invention, unless otherwise stated, the scientific and technical terms used herein have meanings commonly understood by those skilled in the art. Furthermore, the terms related to protein and nucleic acid chemistry, molecular biology, cell and tissue culture, microbiology, immunology and laboratory operation steps used herein are all terms and routine steps widely used in corresponding fields. Meanwhile, in order to better understand the present invention, definitions and explanations of related terms will be provided below.

As used herein, the term “and/or” covers all combinations of items connected by the term, and each combination shall be deemed to have been listed separately herein. For example, “A and/or B” covers “A”, “A and B” and “B”. For example, “A, B and/or C” covers “A”, “B”, “C”, “A and B”, “A and C”, “B and C” and “A and B and C”.

“Ferritin” refers to an iron storage structure composed of a protein shell and an iron inner core. Naturally, the protein shell of the ferritin is a cage protein structure (with an outer diameter of 12 nm and an inner diameter of 8 nm) formed by self-assembling 24 subunits, and the main component of the iron inner core is ferrihydrite. The protein shell of the ferrintin without the iron inner core is referred to as “apoferritin”. The “ferritin” described herein comprises eukaryotic ferritins and prokaryotic ferritins, preferably eukaryotic ferritins, more preferably mammalian ferritins, such as human ferritin. The eukaryotic ferritin generally comprises a heavy chain H subunit and a light chain L subunit. In different tissues and organs of an organism, a ferrintin molecule contains different proportions of H and L subunits. However, “H ferritin (HFn)” formed only by assembling H subunits or “L ferritin (LFn)” formed only by assembling L subunits can also be obtained through recombination.

“Cage protein”, referred to as “nano cage”, refers to a three-dimensional protein structure, that is, a cage structure, which is formed by a plurality of polypeptides (subunits) capable of self assembling and has an internal central cavity. The number of polypeptides (subunits) assembled into cage protein is not specially limited, as long as they can form the cage structure. The cage protein can have a symmetric structure, or an asymmetric structure, which depends on compositions of its subunits. The typical cage protein comprises ferritin/apoferritin.

“Polypeptide”, “peptide” and “protein” can be used interchangeably herein, which refers to a polymer of amino acid residues. This term is suitable for amino acid polymers of artificial chemical analogues in which one or more of amino acid residues are corresponding natural amino acids, and is suitable for polymers of natural amino acids. The term “polypeptide”, “peptide”, “amino acid sequence” and “protein” can also comprise modification forms, including, but not limited to, glycosylation, lipid binding, sulfation, y carboxylation and hydroxylation of glutamic acid residues and ADP ribosylation.

As used herein, “polynucleotide” refers to a macromolecule formed by linking multiple nucleotides via phosphodiester linkages, wherein the nucleotide comprises ribonucleotide and deoxyribonucleotide. The sequence of the polynucleotide of the present invention can be subjected to codon optimization for different host cells (such as Escherichia coli), thereby improving the expression of the polypeptide. Methods for codon optimization are known in the art.

When the phrase “comprise” herein is used for describing the sequence of a protein or nucleic acid, the protein or nucleic acid can be composed of the sequence, or can have additional amino acids or nucleotides at one end or two ends, but still has the activity of the present invention. In addition, those skilled in the art know that methionine encoded by a starting codon at the N terminal of the polypeptide can be retained in some cases (for example when it is expressed by a special expression system), but the function of the polypeptide is not substantially affected. Hence, when a specific polypeptide amino acid sequence is described in specification and claims of the present application, although the polypeptide amino acid sequence may not comprise methionine encoded by a starting codon at the N terminal, at this moment, it also contains the sequence with this methionine. Correspondingly, its coding nucleotide sequence can also comprise the starting codon.

“Sequence identity” between two polypeptides or two polynucleotide sequences refers to percentage of identical amino acids or nucleotides between the sequences. Methods for assessing the level of sequence identity between polypeptide or polynucleotide sequences are known in the art. Sequence identity can be assessed using various known sequence analysis software. For example, sequence identity can be assessed by an on-line alignment tool of EMBL-EBI (https://www.ebi.ac.uk/Tools/psa/). Sequence identity between two sequences can be assessed using default parameters through Needleman-Wunsch algorithm.

As used herein, “expression construct” refers to a vector, such as a recombinant vector, which is suitable for expression of a nucleotide sequence of interest in an organism. “Expression” refers to generation of a functional product. For example, the expression of the nucleotide sequence can refer to transcription of the nucleotide sequence (for example transcribed into mRNA or functional RNA) and/or translation of RNA into a precursor or a mature protein. The “expression vector” of the present invention can be a linear nucleic acid fragment, a circular plasmid, a viral vector, or can be RNA that is translated (such as mRNA). Usually, in the expression construct, the nucleotide sequence of interest is operably linked to a regulatory sequence.

“Regulatory sequence” and “regulatory element” can be interchanged, which refers to a nucleotide sequence which is located at the upstream (5′ non-coding sequence”), middle or downstream (3′ non-coding sequence) and affects the transcription, RNA processing or stability or translation of a sequence of interest. The regulatory sequence can include but is not limited to a promoter, a translation preamble sequence, an intron and a polyadenylation recognition sequence.

As used herein, the term “operably linked” refers to linking the regulatory sequence to a target nucleotide sequence so that the transcription of the target nucleotide sequence is controlled and regulated by the regulatory sequence. The technology for operably linking the regulatory sequence to the target nucleotide sequence is known in the art.

As used herein, “active pharmaceutical ingredient” or “active drug ingredient” or “API (active pharmaceutical ingredient)” refers to a substance which has pharmacological activity or is capable of directly affecting the function of an organism in a drug. Usually, “active pharmaceutical ingredient” does not comprise the drug carrier or an excipient.

“Pharmaceutically acceptable excipeint” used herein refers to any component which has no pharmacological activity and no toxicity used in preparation of a drug product, including but not limited to a disintegrating agent, an adhesive, a filler, a buffer, a tension agent, a stabilizer, an antioxidant, a surfactant or a lubricant.

As used herein, “effective amount” or “therapeutically effective dose” refers to an amount of a substance, a compound, a material or a compound-containing composition that is sufficient to create a curative effect after being administrated to a subject. Therefore, the amount is necessary for preventing, curing, improving, blocking or partially blocking the symptoms of a disease.

### II. Mutant Polypeptide of Ferritin Heavy Chain (H) Subunit

In the present invention, a functional molecule (an antibody molecule, a tracing molecule or a small molecule peptide) is conjugated with a sulfydryl group (SH) on the surface of ferritin through chemical coupling, overcoming the above technical problem generated when a ferritin drug carrier is constructed by fusion.

The wild type human ferritin H subunit has 3 sulfydryl groups respectively located in Loop region between the second α helix and the third α helix (a sulfydryl group of cysteine at position 90 of the wild type human ferritin H subunit), on the third α helix (a sulfydryl group of cysteine at position 102 of the wild type human ferritin H subunit) and near the triple symmetrical axis region of the fourth α helix (a sulfydryl group of cysteine at position 130 of the wild type human ferritin H subunit). However, during conjugation, if there are multiple reaction sites, specific positions of conjugation and reaction ratio of the functional molecule to HFn cannot be controlled.

The inventors found that, by comprising one cysteine only in the loop region (corresponding to amino acids at positions 79-91 of the wild type human ferritin H subunit) of the ferritin H subunit while removing other sulfydryl groups on the surface, each ferritin subunit only retain one chemical conjugation site, which can form a nano protein sphere having 24 conjugation sites on the surface. Through chemical reaction, multiple different functional active molecules or multiple identical functional molecules are coupled to ferritin in a uniform and controllable manner to form a multi-valent multi-effect nano particle that is stable, uniform and suitable for forming drugs, thereby exerting multiple functions such as treatment, diagnosis, prevention and detection.

Therefore, in one aspect, the present invention provides a ferritin heavy chain (H) subunit mutant polypeptide, wherein relative to a wild type ferritin H subunit, the mutant polypeptide comprises one cysteine residue in loop region, the cysteine at a position corresponding to position 102 of SEQ ID NO:1 is substituted, and optionally, the cysteine at a position corresponding to position 130 of SEQ ID NO:1 is substituted. In some embodiments, the loop region corresponds to amino acid residues at positions 79-91 of SEQ ID NO:1. In some embodiments, except one cysteine residue in loop region and/or a cysteine residue at a position corresponding to position 130 of SEQ ID NO:1, the mutant polypeptide does not comprise additional cysteine residues. In some preferred embodiments, the mutant polypeptide does not comprise cysteine residues outside the loop region.

The ferritin H subunit of the present invention includes but is not limited to a mammalian ferritin H subunit, such as a human ferritin H subunit or a horse ferritin H subunit, preferably the human ferritin H subunit. An exemplary wildtype human ferritin H subunit comprises the amino acid sequence of SEQ ID NO:1.

In some embodiments, relative to a wild type ferritin H subunit, the mutant polypeptide comprises a cysteine at a position corresponding to position 90 of SEQ ID NO:1, and the cysteine at a position corresponding to position 102 of SEQ ID NO:1 is substituted, preferably the cysteine at a position corresponding to position 130 of SEQ ID NO:1 is substituted. In some embodiments, relative to the wildtype ferritin H subunit, the mutant polypeptide comprises a cysteine at a position corresponding to position 90 of SEQ ID NO:1, and cysteines at positions corresponding to position 102 and position 130 of SEQ ID NO:1 are substituted. In some embodiments, the cysteines at positions corresponding to position 102 and/or position 130 of SEQ ID NO:1 are substituted by amino acids selected from serine, threonine, asparagine, glutamine, glutamic acid, aspartic acid, lysine, arginine, histidine, alanine and glycine, preferably serine, or amino acids at corresponding positions of a wildtype ferritin light chain (L) subunit polypeptide. The amino acid sequence of an exemplary wildtype ferritin light chain (L) subunit polypeptide is as shown in SEQ ID NO:32.

In some embodiments, relative to the wild type ferritin H subunit, the cysteines of the mutant polypeptide at positions corresponding to position 90 and position 102 of SEQ ID NO:1 are substituted; optionally, the cysteine of the mutant polypeptide at a position corresponding to position 130 of SEQ ID NO:1 is substituted; and the amino acid of the mutant polypeptide at a position corresponding to one of position 79, 80, 81, 82, 83, 84, 85, 86, 87, 88, 89 and 91 of SEQ ID NO:1 is substituted by cysteine. In some embodiments, relative to the wild type ferritin H subunit, the cysteines of the mutant polypeptide at positions corresponding to position 90, 102 and/or 103 of SEQ ID NO:1 are substituted; and the amino acid of the mutant polypeptide at a position corresponding to one of position 79, 80, 81, 82, 83, 84, 85, 86, 87, 88, 89 and 91 of SEQ ID NO:1 is substituted by cysteine. In some embodiments, the cysteines of the mutant polypeptide at positions corresponding to position 90, 102 and/or 103 of SEQ ID NO:1 are substituted by amino acids selected from serine, threonine, asparagine, glutamine, glutamic acid, aspartic acid, lysine, arginine, histidine, alanine and glycine, preferably serine, or amino acids at corresponding positions of a wild type ferritin light chain (L) subunit polypeptide.

In some embodiments, the amino acid residue such as arginine residue (R) of the mutant polypeptide at a position corresponding to position 79 of SEQ ID NO:1 is substituted by cysteine residue (C). In some embodiments, the amino acid residue such as isoleucine residue I of the mutant polypeptide at a position corresponding to position 80 of SEQ ID NO:1 is substituted by cysteine residue. In some embodiments, the amino acid residue such as phenylalanine residue F of the mutant polypeptide at a position corresponding to position 81 of SEQ ID NO:1 is substituted by cysteine residue. In some embodiments, the amino acid residue such as leucine residue L of the mutant polypeptide at a position corresponding to position 82 of SEQ ID NO:1 is substituted by cysteine residue. In some embodiments, the amino acid residue such as glutamine residue Q of the mutant polypeptide at a position corresponding to position 83 of SEQ ID NO:1 is substituted by cysteine residue. In some embodiments, the amino acid residue such as aspartate residue D of the mutant polypeptide at a position corresponding to position 84 of SEQ ID NO:1 is substituted by cysteine residue. In some embodiments, the amino acid residue such as isoleucine residue I of the mutant polypeptide at a position corresponding to position 85 of SEQ ID NO:1 is substituted by cysteine residue. In some embodiments, the amino acid residue such as lysine residue K of the mutant polypeptide at a position corresponding to position 86 of SEQ ID NO:1 is substituted by cysteine residue. In some embodiments, the amino acid residue such as lysine residue K of the mutant polypeptide at a position corresponding to position 87 of SEQ ID NO:1 is substituted by cysteine residue. In some embodiments, the amino acid residue such as proline residue P of the mutant polypeptide at a position corresponding to position 88 of SEQ ID NO:1 is substituted by cysteine residue. In some embodiments, the amino acid residue such as aspartate residue D of the mutant polypeptide at a position corresponding to position 89 of SEQ ID NO:1 is substituted by cysteine residue. In some embodiments, the amino acid residue such as aspartate residue D of the mutant polypeptide at a position corresponding to position 91 of SEQ ID NO:1 is substituted by cysteine residue.

In some specific embodiments, the mutant polypeptide comprises an amino acid sequence selected from one of SEQ ID Nos:2-14 and 28.

In some embodiments, the mutant polypeptide can be assembled into a cage protein and/or can confer the cage protein with an ability of specifically binding to a TfR1 receptor after being assembled into the cage protein.

### III. A Polynucleotide, an Expression Construct, a Host Cell and a Preparation Method of a Ferritin H Subunit Mutant Polypeptide

In another aspect, the present invention provides an isolated polynucleotide, comprising a nucleotide sequence encoding the recombinant ferritin H subunit polypeptide of the present invention.

In some embodiments, the polynucleotide of the present invention comprises for example a nucleotide sequence selected from one of SEQ ID NOs:15-27 and 30.

In another aspect, the present invention provides an expression construct, comprising the polynucleotide of the present invention which is operably linked to an expression regulatory sequence.

Vectors for the expression construct of the present invention comprise those vectors that autonomously replicate in host cells, such as a plasmid vector; also comprise vectors that can be integrated into host cell DNA and replicate together with host cell DNA. Many vectors suitable for the present invention can be commercially available. In a specific embodiment, the expression construct of the present invention is derived from pET22b of Novagen company.

In another aspect, the present invention provides a host cell, comprising the polynucleotide of the present invention or being transformed by the expression construct of the present invention, wherein the host cell can express the ferritin H subunit mutant polypeptide of the present invention.

The host cells for expressing the ferritin H subunit mutant polypeptide of the present invention comprise prokaryotes, yeasts and higher eukaryotic cells. Exemplary prokaryotic hosts comprise bacteria of Escherichia, Bacillus, Salmonella, Pseudomonas and Streptomyces. In a preferred embodiment, the host cell is an Escherichia cell, preferably Escherichia coli. In a specific embodiment of the present invention, the used host cell is a cell of Escherichia coli BL21 (DE3) strain.

The recombinant expression construct of the present invention can be introduced into the host cell through one of many known technologies including but not limited to heat shock transformation, electroporation, DEAE-glucosan transfection, microinjection, liposome-mediated transfection, calcium phosphate precipitation, protoplast fusion, particle bombardment, virus transformation and similar technologies.

In another aspect, the present invention provides a method for producing the ferritin H subunit mutant polypeptide of the present invention, comprising:

a) culturing the host cell of the present invention under the condition of allowing the expression of the polypeptide;

b) obtaining the polypeptide expressed by the host cell from the culture obtained in step a); and

c) optionally further purifying the polypeptide obtained in step b).

However, the ferritin H subunit mutant polypeptide of the present invention can also be obtained by a chemical synthesis method.

### IV. Polypeptide Conjugate

In another aspect, the present invention provides a polypeptide conjugate, comprising the ferritin H subunit mutant polypeptide of the present invention, and a functional moiety conjugated with the sulfydryl group of the ferritin H subunit mutant polypeptide. In some embodiments, the functional moiety is conjugated with the ferritin H subunit mutant polypeptide of the present invention only through a cysteine residue in loop region.

In some embodiments, the functional moiety is selected from a therapeutic molecule, a detectable molecule or a targeting molecule.

The therapeutic molecule includes but is not limited to a small molecule drug, a therapeutic polypeptide or a therapeutic antibody, etc. Exemplary therapeutic small molecule includes but is not limited to a toxin, an immunomodulator, an antagonist, an apoptosis inducer, a hormone, a radiopharmaceutical, an antiangiogenic agent, siRNA, a cytokine, a chemokine, a prodrug, a chemotherapy drug, etc. In some specific embodiments, therapeutic molecule is 7-ethyl-10-hydroxycamptothecin (SN38). The structural formula of SN38 is as shown in the following formula:

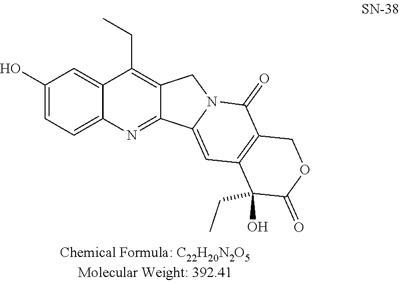 [SMILES: CCc1c2c(nc3ccc(O)cc13)-c1cc3c(c(=O)n1C2)COC(=O)[C@]3(O)CC]

The detectable molecule includes but is not limited to a fluorescent molecule, a luminous chemical, an enzyme, an isotope, a label, etc.

The targeting molecule includes but is not limited to a targeting antibody, a specific receptor ligand, etc. For example, the targeted molecule can be an antibody specifically targeting a tumor antigen.

In some embodiments, the functional moiety is conjugated with the ferritin H subunit mutant polypeptide through a linker.

In some embodiments, the polypeptide conjugate can be assembled into a cage protein and/or can confer the cage protein with an ability of specifically binding to the TfR1 receptor after being assembled into the cage protein.

In some embodiments, the polypeptide conjugate is an isolated polypeptide conjugate, which for example is not assembled into the cage protein. In some embodiments, the polypeptide conjugate is contained in the cage protein.

### V. Cage Protein

Since the self-assembly ability and/or receptor binding ability of the wild type ferritin H subunit are retained, the ferritin H subunit mutant polypeptide of the present invention can be independently assembled into a cage protein (i.e., H ferritin/apoferritin) in an appropriate medium, and can also form the cage protein with a ferritin L subunit or other ferritin H subunits or other self-assembling polypeptides, and can confer the cage protein with a specific targeting ability.

Therefore, in another aspect, the present invention provides a cage protein, comprising at least one ferritin H subunit mutant polypeptide of the present invention and/or at least one polypeptide conjugate of the present invention.

Exemplary cage protein can comprise for example 1, 2, 3, 4, 5, 6, 7, 8, 9, 10, 11, 12, 13, 14, 15, 16, 17, 18, 19, 20, 21, 22, 23, 24, 36 or 48 ferritin H subunit mutant polypeptides of the present invention and/or for example 1, 2, 3, 4, 5, 6, 7, 8, 9, 10, 11, 12, 13, 14, 15, 16, 17, 18, 19, 20, 21, 22, 23, 24, 36 or 48 polypeptide conjugates of the present invention. In some preferred embodiments, the cage protein comprises 24 ferritin H subunit mutant polypeptides of the present invention and/or 24 polypeptide conjugates of the present invention.

In some embodiments, the cage protein only comprises the ferritin H subunit mutant polypeptide of the present invention and/or polypeptide conjugate of the present invention, for example, only comprises the polypeptide conjugate of the present invention. For example, in some preferred embodiments, the cage protein is formed by assembling 24 polypeptide conjugates of the present invention.

In some embodiments, the cage protein comprises a plurality of polypeptide conjugates of the present invention which comprise identical or different functional moieties.

In some embodiments, the cage protein also comprises ferritin L subunits. In some embodiments, the cage protein comprises at least one ferritin L subunit mutant polypeptide of the present invention and at least one ferritin L subunit, preferably, a ratio range of the ferritin L subunit mutant polypeptide to the ferritin L subunit can be for example 1:23-23:1.

In some embodiments, the cage protein does not comprise ferritin L subunit.

### VI. Conjugation Method

Many methods for conjugating a functional molecule with a protein through a sulfydryl group are known in the art, all of which can be applied to the present invention. Those skilled in the art can determine proper conjugation methods according to specific functional molecules and selected linkers. For exemplary methods, please refer to Moon, S. J., et al., Antibody conjugates of 7-ethyl-10-hydroxycamptothecin (SN-38) for targeted cancer chemotherapy. J Med Chem, 2008. 51(21): p. 6916-26.

In one aspect, the present invention provides a method for preparing the cage protein of the present invention, the cage protein comprising at least one polypeptide conjugate of the present invention, and the method comprising:

a) conjugating a functional molecule to a disassembled ferritin H subunit mutant polypeptide of the present invention, and

b) assembling the ferritin H subunit mutant polypeptide conjugated with the functional molecule into a cage protein.

Preferably, this method is suitable for the ferritin H subunit mutant polypeptide of the present invention which comprises one cystine only in loop region.

In some embodiments, the functional molecule is SN38. In some embodiments, the step a) comprises contacting the compound of the following formula with the disassembled ferritin H subunit mutant polypeptide of the present invention.

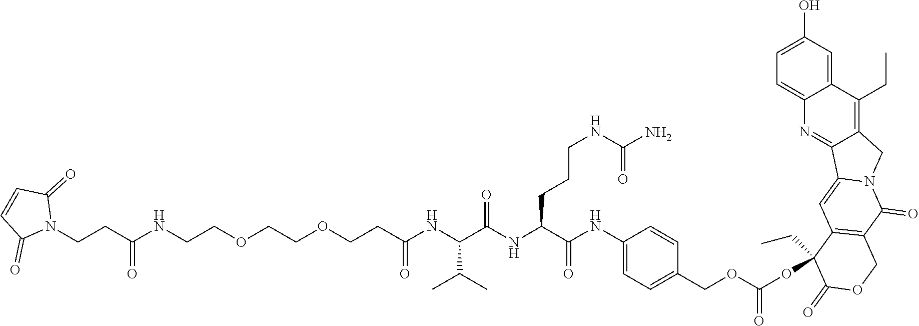 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

In one aspect, the present invention provides a method for preparing a cage protein, the cage protein comprising at least one polypeptide conjugate of the present invention, and the method comprising:

a) providing a cage protein comprising at least one ferritin H subunit mutant polypeptide, and

b) conjugating the functional molecule to the ferritin H subunit mutant polypeptide of the present invention in the cage protein.

This method is suitable for the ferritin H subunit mutant polypeptide of the present invention which comprises a cystine at a position corresponding to position 130 of SEQ ID NO:1 in addition to one cystine in loop region, and is also suitable for the ferritin H subunit mutant polypeptide of the present invention which only comprises one cystine in loop region.

In some embodiments, the functional molecule is SN38. In some embodiments, the step b) comprises contacting the compound of the following formula with the cage protein.

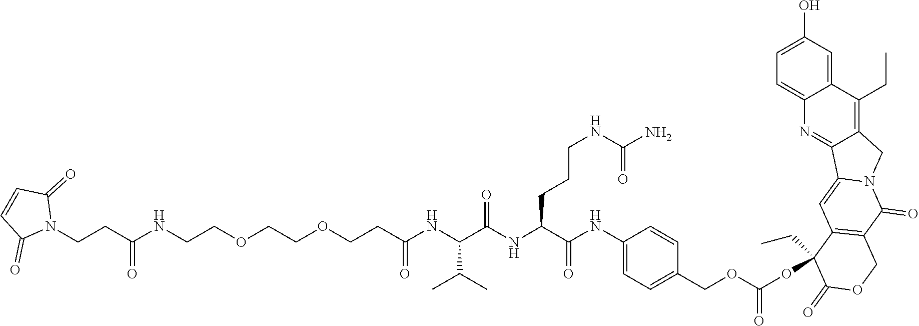 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

### VII. Cage Protein-API Complex

In another aspect, the present invention provides a cage protein-API complex, wherein the cage protein-API complex comprises the cage protein of the present invention, and an active pharmaceutical ingredient (API) loaded inside the cage protein.

In some embodiments, the cage protein in the complex comprises the polypeptide conjugate of the present invention, and the conjugate comprises the ferritin H subunit mutant polypeptide of the present invention and a therapeutic molecule. The cage protein of the present invention that is conjugated with a therapeutic molecule can simultaneously deliver different therapeutically effective components in two different manners.

In some embodiments, the cage protein in the complex comprises the polypeptide conjugate of the present invention, and the conjugate comprises the ferritin H subunit mutant polypeptide of the present invention and a detectable molecule. The cage protein of the present invention that is conjugated with a detectable molecule can be used for monitoring (for example real-time monitoring) the delivery of a drug.

In some embodiments, the cage protein in the complex comprises the polypeptide conjugate of the present invention, the conjugate comprises the ferritin H subunit mutant polypeptide of the present invention and a targeting molecule. The cage protein of the present invention that is conjugated with the targeting molecule can target additional therapeutic targets in vivo.

There is no special limitation on the active pharmaceutical ingredient (API) loaded inside the cage protein, as long as it is suitable for loading into the cage protein of the present invention, for example, the API does not damage the cage structure of the cage protein and/or its size is suitable for being accommodated by the cage structure. The examples of the API include but are not limited to alkylating agents, platinum, antimetabolic drugs, tumor antibiotics, natural extracts, hormones, radiopharmaceuticals, neurotransmitters, dopamine receptor agonists, neurocentral anticholinergics, choline receptor agonist drugs, y secretase inhibitors, antioxidants and anesthetics.

### VIII. Pharmaceutical Composition and Use Thereof

In another aspect, the present invention provides a pharmaceutical composition, comprising the ferritin H subunit mutant polypeptide of the present invention, the polypeptide conjugate of the present invention, the cage protein of the present invention and/or the cage protein-PAI complex of the present invention, and a pharmaceutically acceptable excipient.

In some embodiments, the pharmaceutical composition comprises the ferritin H subunit mutant polypeptide of the present invention or the polypeptide conjugate of the present invention and optionally an effective amount of API, wherein the ferritin H subunit mutant polypeptide and the polypeptide conjugate of the present invention are provided in a form of not being assembled into a cage protein. The ferritin H subunit mutant polypeptide or the polypeptide conjugate can be self-assembled into the cage protein or the cage protein-API complex in vitro or after delivery to a body under suitable conditions.

In some embodiments, where the polypeptide conjugate of the present invention comprises a therapeutic molecule, the pharmaceutical composition comprising the polypeptide conjugate of the present invention may comprise no additional API.

Diseases that can be treated and/or prevented by using the ferritin H subunit mutant polypeptide, the polypeptide conjugate, the cage protein, the cage protein-API complex and/or the pharmaceutical composition of the present invention depend on the therapeutic molecule or API contained therein. Furthermore, the cage protein of the present invention is especially suitable for treating tumors or brain diseases due to its tumor targeting ability and blood brain barrier penetration ability. In addition, if the polypeptide conjugate of the present invention comprises a targeting molecule, depending on a target of this targeting molecule, the ferritin H subunit mutant polypeptide, the polypeptide conjugate, the cage protein, the cage protein-API complex and/or the pharmaceutical composition of the present invention can also be used for other diseases.

Examples of brain diseases include, but are not limited to, for example brain tumor, Alzheimer's disease, Parkinson's disease, stroke, epilepsy, Huntington's disease and amyotrophic lateral sclerosis. Examples of tumors include, but are not limited to, for example colorectal cancer, lung cancer, breast cancer, ovarian cancer, melanoma, gastric cancer, pancreatic cancer, bladder cancer, kidney cancer, prostate cancer, and various hematopoietic system cancers such as Hodgkin's disease, non-Hodgkin's lymphoma and leukemia.

In another aspect, the present invention provides use of the ferritin H subunit mutant polypeptide, the polypeptide conjugate, the cage protein, the cage protein-API complex and/or the pharmaceutical composition of the present invention in preparation of a medicine. In some embodiments, the medicine is for example used for treating a tumor or a brain disease.

In another aspect, the present invention provides a method for treating and/or preventing a disease in a subject, the method comprising administrating an effective amount of ferritin H subunit mutant polypeptide, polypeptide conjugate, cage protein, cage protein-API complex and/or pharmaceutical composition of the present invention to the subject. The disease is as defined above, preferably is a tumor or a brain disease.

The polypeptide of the present invention, the ferritin H subunit mutant polypeptide, the polypeptide conjugate, the cage protein, the cage protein-API complex and/or the pharmaceutical composition of the present invention can be administrated by any appropriate methods known by persons of ordinary skill in the art (see for example, Remington: The Science and Practice of Pharmacy,” edition 21, 2005). The pharmaceutical composition can be administrated for example in an intravenous, intramuscular, intraperitoneal, cerebrospinal, subcutaneous, intraarticular, synovial, intrathecal, oral, local or inhalation route.

### IX. Methods for Preparing a Cage Protein-API Complex

In another aspect, the present invention provides a method for preparing the cage protein-API complex of the present invention, the method comprising contacting the ferritin H subunit mutant polypeptide of the present invention, the polypeptide conjugate of the present invention and/or the cage protein of the present invention with an API, so as to obtain the cage protein-API complex.

In some embodiments, the method comprises:

a) contacting a disassembled cage protein of the present invention with the API; and

b) reassembling the cage protein so as to obtain the cage protein-API complex.

As used herein, “disassembled” refers to a process that under certain conditions, the tightly closed spherical structure of the cage protein is opened, so that all or a part of its subunits are separated from each other, the conditions are for example protein denaturation conditions, such as a buffer solution with high concentration of urea.

As used herein, “reassembling” refers to a process of self-assembling a disassembled cage protein, namely isolated subunits, into a cage protein again by altering conditions for example changing into physiological compatibility conditions. In the process of reassembling the cage protein, API will be coated inside the cage protein, thereby forming the cage protein-API complex. The physiological compatibility condition is for example a physiological buffer solution.

In some embodiments, the method also comprises a step of disassembling the cage protein of the present invention prior to the step a). In some embodiments, the cage protein of the present invention is disassembled in the presence of high-concentration (for example at least 6 M, preferably 8 M) urea. In some embodiments, the cage protein is reassembled by reducing the concentration of urea step by step (for example by gradient dialysis).

In some embodiments, the method comprises:

a) contacting the cage protein of the present invention with API under the non-disassembling condition, thereby allowing API to bind to the cage protein and/or to be loaded to the internal central cavity of the cage protein, and

b) obtaining the cage protein-API complex.

In some embodiments, the non-disassembling condition comprises placing the cage protein and API in a physiologically acceptable buffer solution. Proper physiologically acceptable buffer solutions include but are not limited to a PBS solution, normal saline, pure water, a HEPES buffer solution, etc.

In some embodiments, API binds to the cage protein through non-covalent or covalent interaction. The non-covalent interaction includes for example Van der Waals force, a hydrogen bond, an ionic bond, etc. The covalent interaction includes reaction with a free amino group and carboxyl group on the surface of the cage protein, such as condensation reaction.

In some embodiments, API is shuttled to the internal central cavity of the cage protein by passive diffusion. API can enter the internal central cavity of the cage protein by diffusion without disassembling the cage protein by placing the cage protein and API into a physiologically acceptable buffer solution.

### EXAMPLES

The present invention will be further understood by reference to some specific examples given herein, and these embodiments are only for illustrating the present invention but not intended to limit the scope of the present invention. Obviously, multiple modifications and changes can be made to the present invention without departing from the essence of the present invention. Thus, these modifications and changes are similarly included within the scope claimed by the present application.

### Example 1 Design and Expression of Ferritin H Subunit Mutant

Based on a wild type ferrtin H subunit (SEQ ID NO:1), the inventor designed a mutant comprising one Cys for conjugation in Loop region. The three natural Cys of the ferrtin H subunit, i.e., amino acid residues at position 90, position 102 and position 130, were mutated into Ser. Meanwhile, 13 amino acids in Loop region (positions 79-91) were successively and respectively mutated into Cys. Specific design is as shown in Table 1.

TABLE 1
HFn mutation design scheme

| Number of mutant | Mutation site | | | | SEQ ID NO |
|---|---|---|---|---|---|
| HFn-WT | C90 | C102 | C130 | | 1 |
| HFn-Mt-1 | C90S | C102S | C130S | R79C | 2 |
| HFn-Mt-2 | C90S | C102S | C130S | I80C | 3 |
| HFn-Mt-3 | C90S | C102S | C130S | F81C | 4 |
| HFn-Mt-4 | C90S | C102S | C130S | L82C | 5 |
| HFn-Mt-5 | C90S | C102S | C130S | Q83C | 6 |
| HFn-Mt-6 | C90S | C102S | C130S | D84C | 7 |
| HFn-Mt-7 | C90S | C102S | C130S | I85C | 8 |
| HFn-Mt-8 | C90S | C102S | C130S | K86C | 9 |
| HFn-Mt-9 | C90S | C102S | C130S | K87C | 10 |
| HFn-Mt-10 | C90S | C102S | C130S | P88C | 11 |
| HFn-Mt-11 | C90S | C102S | C130S | D89C | 12 |
| HFn-Mt-12 | C90 | C102S | C130S | | 13 |
| HFn-Mt-13 | C90S | C102S | C130S | D91C | 14 |

Gene sequence design was performed according to the amino acid sequence of the designed mutant HFn and codon preference of host bacteria. The nucleotide sequences are shown in SEQ ID NOs:15-28.

The commonly used vector pET-30a(+) for expressing foreign proteins in Escherichia coli was selected and showed kanamycin resistance (Kan+), and Nde I and Hind III restriction sites were selected to allow a target gene to be inserted therein. The successful construction of the expression vector was confirmed by restriction enzyme map and gene sequencing.

E. coli BL21 (DE3) was selected as a host bacterium, recombinant plasmids containing target genes were transformed into the competent cell of the host bacterium, positive clones were screened through a resistance plate containing kanamycin to determine recombinant strains.

The recombinant strains were inoculated into a 750 mL LB culture medium/2 L shake bottle at 1‰ under the conditions of 37° C. and 220 rpm. After inoculation, the strains were cultured for about 7 h under the conditions of 37° C. and 220 rpm, IPTG with a fmal concentration of 1 mM was added to induce the expression of a target protein, wherein the culture conditions during the induction include 30° C. and 220 rpm, and then bacterial sludge was collected by centrifugation after inducing for 5-6 h.

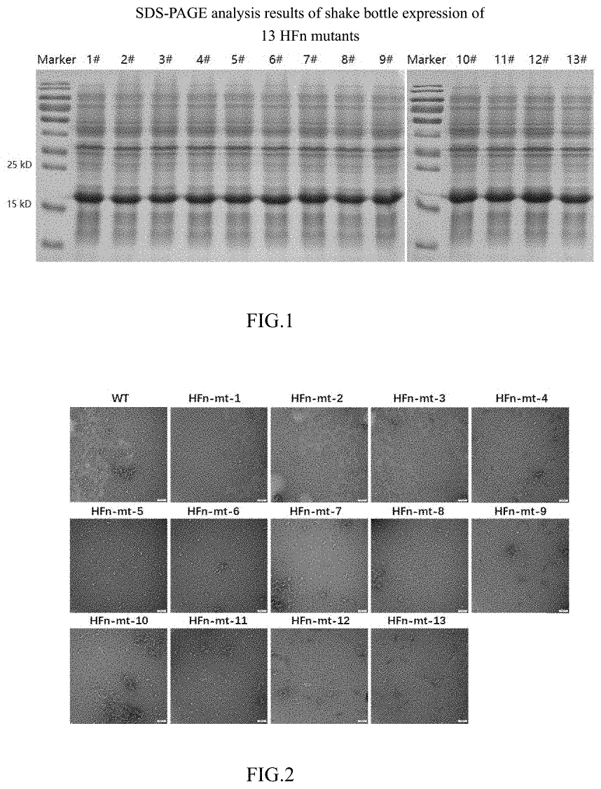

9 mL of bacterial solution was centrifuged for 10 min at 5000 r/min, the supernatant was discarded, 1.5 mL of 20 mM Tris-HCl was added for resuspension, ultrasonication was performed for 2 min under the conditions of turning on for 2 s and turning off for 3 s at 150 Hz, the lysate was centrifuged for 30 min at 5000 r/min, the supernatant was taken and detected by SDS-PAGE, wherein a loading amount was 10 μl (sample: Loading buffer=1:1). An expression result of each protein is as shown in FIG. 1.

The protein purification method comprises the following steps: after Escherichia coli cells that had been subjected to induced expression was resuspended with a 20 mM Tris (pH8.0) buffer solution, the cells were broken by ultrasonic lysis; Escherichia coli cell fragments were removed by centrifugation (1500 rpm, 10 min); the supernatant was heated for 15 minutes at 72° C.; unwanted proteins were precipitated, and the precipitate was removed by centrifugation; the supernatant was isolated and purified on an exclusion chromatography Superdex 200 pg column; purity was identified by SDS-PAGE; the concentration of the protein was determined by BCA. The protein purification effect was detected by SEC-HPLC.

### Example 2 Characterization of Ferritin H Subunit Mutant

2.1 TEM Results of Mutant HFn

A protein sample (20 μL, 0.1 mg/mL) was dropwise added into a treated copper mesh and dyed with 1% uranyl acetate for 1 minute, and then imaged with JEM-1400 80 kv TEM (JEOL, Japan). A transmission electron microscope result (FIG. 2) shows that both the mutated H subunit polypeptide and the wild type H subunit polypeptide can form an uniform and regular cage protein structure with a diameter of about 12-16 nm.

2.2 DLS Particle Size Detection of Mutant HFn

The particle size of a sample was detected using Nano ZSE Nanosizer (Malvern, UK). Parameters were set as follows: Material was Protein, Dispersant was a pH 8.0 50 mM Tris buffer solution. An automatic mode was selected for scanning, and each sample was scanned three times. The scanning results were averaged.

The samples were all stored in pH 8.0 50 mM Tris buffer solution. Concentrations of proteins in specific samples are as shown in Table 2 below.

TABLE 2
DLS particle size detection results of mutant HFn

| | Concentration | Particle size | |
|---|---|---|---|
| Sample | (mg/mL) | (d, nm) | PDI |
| HFn-Mt-1 | 3.67 | 13.91 ± 0.345 | 0.025 ± 0.011 |
| HFn-Mt-2 | 3.58 | 16.63 ± 0.635 | 0.178 ± 0.007 |
| HFn-Mt-3 | 1.88 | 19.81 ± 0.478 | 0.179 ± 0.005 |
| HFn-Mt-4 | 3.99 | 15.43 ± 0.160 | 0.153 ± 0.006 |
| HFn-Mt-5 | 2.57 | 14.95 ± 1.135 | 0.122 ± 0.043 |
| HFn-Mt-6 | — | — | — |
| HFn-Mt-7 | 3.77 | 14.65 ± 0.667 | 0.112 ± 0.012 |
| HFn-Mt-8 | 2.25 | 15.52 ± 0.416 | 0.161 ± 0.005 |
| HFn-Mt-9 | 3.18 | 13.92 ± 0.288 | 0.046 ± 0.013 |
| HFn-Mt-10 | 1.97 | 15.27 ± 0.497 | 0.151 ± 0.011 |
| HFn-Mt-11 | — | — | — |
| HFn-Mt-12 | 3.63 | 14.17 ± 0.397 | 0.039 ± 0.017 |
| HFn-Mt-13 | 2.67 | 13.94 ± 0.301 | 0.029 ± 0.007 |

2.3 Zeta Potential of Mutant HFn in Loop Region

The samples are in the same batch as the above samples whose particle sizes were measured, and diluted 10 times by volume with pH 8.0 50 mM Tris buffer solution before detection.

The Zeta potential of the sample was detected using Nano ZSE Nanosizer (Malvern, UK). Parameters were set as follows: Material was Protein, Dispersant was 50 mM Tris, an automatic mode was selected for scanning, and each sample was scanned three times. The scanning results were averaged.

TABLE 3
Zeta potential detection results of mutant HFn

| Sample | Zeta (mV) |
|---|---|
| HFn-Mt-1 | −17.9 ± 3.61 |
| HFn-Mt-2 | −14.7 ± 3.41 |
| HFn-Mt-3 | −11.2 ± 5.35 |
| HFn-Mt-4 | −15.0 ± 3.56 |
| HFn-Mt-5 | −8.05 ± 1.77 |
| HFn-Mt-6 | — |
| HFn-Mt-7 | −15.4 ± 3.47 |
| HFn-Mt-8 | −17.0 ± 1.12 |
| HFn-Mt-9 | −19.2 ± 1.56 |
| HFn-Mt-10 | −9.40 ± 1.74 |
| HFn-Mt-11 | — |
| HFn-Mt-12 | −22.9 ± 1.41 |
| HFn-Mt-13 | −20.6 ± 0.153 |

2.4 TfR1 Binding Activity of Mutant HFn

Each group of ferritin was diluted to 1 mg/ml with a coating solution (carbonate buffer solution, pH 9.0), the diluted samples were evenly mixed, then added into an ELISA plate according to experimental design in an amount of 100 μl/well, each sample corresponded to three multiple wells, and then the ELISA plate was placed in a refrigerator at 4° C. overnight. Then, the ELISA plate was washed three times with 1×PBST and twice with 1×PBS. A blocking solution (5% skim milk powder) was added in an amount of 300 μL/well for blocking. The samples were incubated for 2 h in an incubator at 37° C. Then, the ELISA plate was washed three times with 1×PBST and twice with 1×PBS. TFR1 (human source) was diluted into 2 μg/mL (1:100) with a protein stabilizer (purchased from Huzhou Yingchuang Biotechnology Co., Ltd, PR-SS-002), and then added in an amount of 100 μL/well. The samples were incubated for 2 h in an incubator at 37° C. The ELISA plate was washed three times with 1×PBST and twice with 1×PBS. An anti-TFR1 antibody (mouse source) (purchased from Beijing Yiqiao Shenzhou Technology Co., Ltd: 11020-MM02) was diluted to 1 μg/mL (1:1000) with the protein stabilizer and then added in an amount of 100 μL/well, and incubated for 1 h in an incubator at 37° C. The ELISA plate was washed three times with 1×PBST and twice with 1×PBS. Anti-mouse IgG was diluted with an HRP coupling stabilizer (1:5000), and then added in an amount of 100 μL/well. The samples were incubated for 1 h in an incubator at 37° C. The ELISA plate was washed three times with 1×PBST and three times with 1×PBS. A TMB one-step developing solution was added in the dark in an amount of 100 μL/well, and then OD 652 nm was immediately determined by ELIASA. Original data was analyzed by Graphpad 6.0 software, time points 15 minutes and 30 minutes were selected to plot a bar graph, the ordinate was an absorption peak value at 652 nm, the abscissa was the coating concentration of the H ferritin (HFn) sample. BSA and L ferritin protein (LFn) without binding activity were used as control.

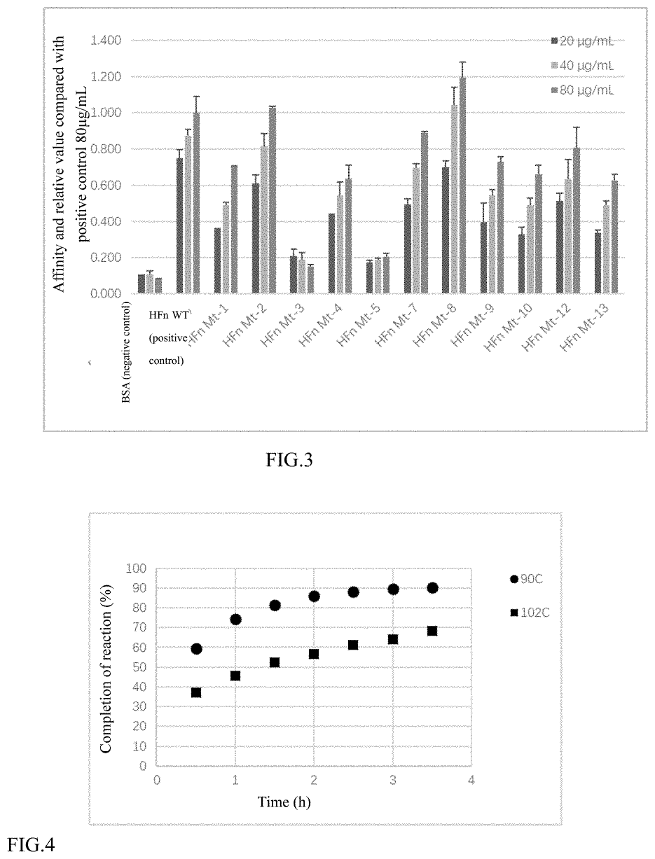

The results are as shown in FIG. 3, showing that by comparing the receptor binding activity of the ferritin with cystine mutation to that of control (HFn-WT), the affinity of HFn-Mt-3 and HFn-Mt-5 completely disappears, the affinity of HFn-Mt-2 and HFn-Mt-8 is equivalent to the affinity of HFn-WT at low concentration, and at high concentration, the affinity of HFn-Mt-8 is slightly higher than that of a wild type. Generally, except for HFn-Mt-3 and HFn-Mt-5, with the increase of concentration, all the mutants retain partial affinity which is lower than that of the wild type. HFn-Mt-2 and HFn-Mt-8 can be considered as retaining the affinity similar to that of the wild type.

### Example 3 PEG Conjugate of Ferritin H Subunit Mutant

3.1 PEG Modification of HFn

The ferririn prepared in example 1 was concentrated to 2 ml at 3500 rpm using a 100 k ultrafiltration centrifuge tube. AKTA was used to change the ferritin solution into conjunction buffer (10 mM PB (pH=6.5)). The ferritin was concentrated at the rotation speed of 3500 rpm using a 100 k ultrafiltration centrifuge tube. The concentrations of the proteins were detected using nanodrop, that is, the concentration of ferritin HFn-Mt-1 was 38.98 mg/ml; the concentration of HFn-Mt-10 was 19.45 mg/ml; the concentration of HFn-Mt-12 was 31.47 mg/ml.

Preparation of PEG solution: 5 mg of Mal-PEG2-CH2-CH2-NHBOC was weighed and dissolved into 5 ml of conjugation buffer to obtain a 1 mg/ml Mal-PEG2-CH2-CH2-NHBOC solution; 4 mg of Mal-PEGS-OCH3 (Mal-mPEG-350 Da) was weighed and dissolved into 4 ml of conjugation buffer to obtain a 1 mg/ml Mal-PEGS-OCH3 solution; 8 mg of Mal-PEG24-OCH3(Mal-mPEG-1000 Da) was weighed and dissolved into 8 ml of conjugation buffer to obtain a 1 mg/ml Mal-PEG24-OCH3 solution.

Linkage reaction of HFn mutant and PEG: different concentrations of PEG solutions previously prepared were added into an HFn mutant solution so that the final concentration of the HFn mutant was 5 mg/ml. In different reaction systems, molar ratios of PEG to HFn were respectively 2:1, 8:1 and 24:1, and each dosing ratio was set for three parallel samples. The samples were evenly vibrated and reacted overnight. Meanings of numbers of samples with different PEG repetitive units and dosing molar ratios are explained in Table 4.

The reaction solution was changed into 10 mM PB (pH=6.5) using a 3K ultrafiltration membrane at the rotation speed of 10000 rpm, unreacted PEG was removed, the remained reaction solution was centrifuged in batch until the content of the reaction solution was less than 3%. The concentrations of the proteins were detected by nanodrop.

TABLE 4

| | | PEG repetitive | PEG-mutant |
|---|---|---|---|
| Name | Mutant | unit | molar ratio |
| HFn-Mt-1 | HFn-Mt-1 | — | — |
| HFn-Mt-1-2-1 | HFn-Mt-1 | 2 | 2:1 |
| HFn-Mt-1-2-2 | HFn-Mt-1 | 2 | 8:1 |
| HFn-Mt-1-2-3 | HFn-Mt-1 | 2 | 24:1 |
| HFn-Mt-1-8-1 | HFn-Mt-1 | 8 | 2:1 |
| HFn-Mt-1-8-2 | HFn-Mt-1 | 8 | 8:1 |
| HFn-Mt-1-8-3 | HFn-Mt-1 | 8 | 24:1 |
| HFn-Mt-1-24-1 | HFn-Mt-1 | 24 | 2:1 |
| HFn-Mt-1-24-2 | HFn-Mt-1 | 24 | 8:1 |
| HFn-Mt-1-24-3 | HFn-Mt-1 | 24 | 24:1 |
| HFn-Mt-9 | HFn-Mt-9 | — | — |
| HFn-Mt-9-2-1 | HFn-Mt-9 | 2 | 2:1 |
| HFn-Mt-9-2-2 | HFn-Mt-9 | 2 | 8:1 |
| HFn-Mt-9-2-3 | HFn-Mt-9 | 2 | 24:1 |
| HFn-Mt-9-8-1 | HFn-Mt-9 | 8 | 2:1 |
| HFn-Mt-9-8-2 | HFn-Mt-9 | 8 | 8:1 |
| HFn-Mt-9-8-3 | HFn-Mt-9 | 8 | 24:1 |
| HFn-Mt-9-24-1 | HFn-Mt-9 | 24 | 2:1 |
| HFn-Mt-9-24-2 | HFn-Mt-9 | 24 | 8:1 |
| HFn-Mt-9-24-3 | HFn-Mt-9 | 24 | 24:1 |

The names of other mutants are analogized under the role in the above table.

3.2 Test Method and Result of Binding Affinity of Mutant HFn-PEG to Tfr-1 Receptor

Each group of ferritin prepared in 3.1 was diluted to 80, 40, 20, 10, 5, 2.5 and 1.25 μg/mL using a coating solution (carbonate buffer, pH 9.0), the diluted samples were evenly mixed, and then added into an ELISA plate in an amount of 100 μL/well according to experimental design, each sample corresponded to three multiple wells, and then the ELISA plate was placed in a refrigerator at 4° C. for overnight. Then, the ELISA plate was washed three times with 1×PBST, and twice with 1×PBST. A blocking solution (5% skim milk powder) was added in an amount of 300 μL/well for blocking. The samples were incubated for 2 h in an incubator at 37° C. Then, the ELISA plate was washed three times with 1×PBST, and twice with 1×PBST. TFR1 (human source) was diluted into 2 μg/mL (1:100) with a protein stabilizer (purchased from Huzhou Yingchuang Biotechnology Co., Ltd, PR-SS-002), and then added in an amount of 100 μL/well. The samples were incubated for 2 h in an incubator at 37° C. The ELISA plate was washed three times with 1×PBST, and three times with 1×PBST. An anti-TFR1 antibody (mouse source) (purchased from Beijing Yiqiao Shenzhou Technology Co., Ltd: 11020-MM02) was diluted to 1 μg/mL (1:1000) with a protein stabilizer and then added in an amount of 100 μL/well, and incubated for 1.5 h in an incubator at 37° C. The ELISA plate was washed three times with 1×PBST, and three times with 1×PBST. Anti-mouse IgG was diluted with an HRP coupling stabilizer (1:5000), and then added in an amount of 100 μL/well. The samples were incubated for 0.5 h in an incubator at 37° C. The ELISA plate was washed three times with 1×PBST. A TMB one-step developing solution was added in the dark in an amount of 100 μL/well, and then OD 652 nm was immediately determined by EIASA. Original data was analyzed by Graphpad 6.0 software, a curve graph was plotted, the ordinate was an absorption peak value at 652 nm, and the abscissa was the coating concentration of the H ferritin (HFn) sample. BSA and L ferritin protein (LFn) without binding activity were used as control.

The binding affinity of PEG modified HFn-Mt-1, HFn-Mt-9, HFn-Mt-10, HFn-Mt-12 and HFn-Mt-13 to Tfr-1 receptor was tested. The results are as shown in Tables 5-9.

TABLE 5
Binding affinity result of HFn-Mt-1 to Tfr-1

| | | | HFn- | HFn- | HFn- | IIFn- | HFn- | HFn- | HFn- | HFn- | HFn- |
|---|---|---|---|---|---|---|---|---|---|---|---|
| Concentration | HFn- | HFn- | Mt-1- | Mt-1- | Mt-1- | Mt-1- | Mt-1- | Mt-1- | Mt-1- | Mt-1- | Mt-1- |
| (μg/ml) | WT | Mt-1 | 2-1 | 2-2 | 2-3 | 8-1 | 8-2 | 8-3 | 24-1 | 24-2 | 24-3 |
| 80 | 7.474 | 3.973 | 4.631 | 3.807 | 2.205 | 5.756 | 5.526 | 2.822 | 6.703 | 4.966 | 1.339 |
| 40 | 6.130 | 2.696 | 2.779 | 2.680 | 1.520 | 3.527 | 4.513 | 2.683 | 7.114 | 4.368 | 1.418 |
| 20 | 5.092 | 1.847 | 1.922 | 2.302 | 1.262 | 2.257 | 3.500 | 2.172 | 6.868 | 4.273 | 1.216 |
| 10 | 3.260 | 1.191 | 1.340 | 1.674 | 1.104 | 1.895 | 2.938 | 1.755 | 5.819 | 2.272 | 1.006 |
| 5 | 3.856 | 0.908 | 0.980 | 1.353 | 0.954 | 1.186 | 2.373 | 1.558 | 4.690 | 1.236 | 0.794 |
| 2.5 | 3.469 | 0.844 | 0.951 | 1.052 | 0.894 | 0.900 | 1.527 | 1.153 | 2.855 | 1.165 | 0.881 |
| 1.25 | 2.623 | 0.898 | 0.906 | 0.911 | 0.925 | 1.004 | 1.140 | 0.988 | 2.140 | 1.039 | 1.081 |

TABLE 6
Binding affinity results of HFn-Mt-9 to Tfr-1

| | | | HFn- | HFn- | HFn- | HFn- | HFn- | HFn- | HFn- | HFn- | HFn- |
|---|---|---|---|---|---|---|---|---|---|---|---|
| Concentration | HFn- | HFn- | Mt-9- | Mt-9- | Mt-9- | Mt-9- | Mt-9- | Mt-9- | Mt-9- | Mt-9- | Mt-9- |
| (μg/ml) | WT | Mt-9 | 2-1 | 2-2 | 2-3 | 8-1 | 8-2 | 8-3 | 24-1 | 24-2 | 24-3 |
| 80 | 5.917 | 3.245 | 3.125 | 3.537 | 4.037 | 3.866 | 4.813 | 4.496 | 4.449 | 4.922 | 5.465 |
| 40 | 5.359 | 2.195 | 2.102 | 1.993 | 2.326 | 2.125 | 2.890 | 2.947 | 3.046 | 4.130 | 5.014 |
| 20 | 4.699 | 0.621 | 1.522 | 1.523 | 1.622 | 1.429 | 1.880 | 2.678 | 2.227 | 3.149 | 3.991 |
| 10 | 3.087 | 1.253 | 1.147 | 1.103 | 1.283 | 0.944 | 1.383 | 1.533 | 1.591 | 1.859 | 2.241 |
| 5 | 3.257 | 1.024 | 0.952 | 0.997 | 1.126 | 1.067 | 1.055 | 1.082 | 1.090 | 1.111 | 1.317 |
| 2.5 | 2.838 | 1.125 | 0.859 | 0.883 | 1.039 | 0.994 | 1.050 | 0.827 | 0.900 | 1.004 | 1.102 |
| 1.25 | 2.840 | 0.896 | 0.949 | 1.450 | 0.960 | 0.858 | 0.785 | 0.879 | 0.855 | 0.820 | 1.043 |

TABLE 7
Binding affinity results of HFn-Mt-10 to Tfr-1

| | | | HFn- | HFn- | HFn- | HFn- | HFn- | HFn- | HFn- | HFn- | HFn- |
|---|---|---|---|---|---|---|---|---|---|---|---|
| | HFn- | HFn- | Mt-10- | Mt-10- | Mt-10- | Mt-10- | Mt-10- | Mt-10- | Mt-10- | Mt-10- | Mt-10- |
| Concentration | WT | Mt-10 | 2-1 | 2-2 | 2-3 | 8-1 | 8-2 | 8-3 | 24-1 | 24-2 | 24-3 |
| 80 | 7.818 | 5.941 | 5.890 | 3.296 | 4.412 | 6.766 | 5.223 | 2.981 | 6.901 | 4.185 | 0.933 |
| 40 | 5.247 | 3.487 | 3.903 | 3.333 | 2.569 | 4.137 | 3.906 | 2.065 | 5.765 | 3.745 | 0.893 |
| 20 | 4.005 | 2.033 | 2.510 | 2.069 | 1.799 | 3.231 | 2.845 | 1.664 | 4.758 | 2.984 | 1.333 |
| 10 | 2.854 | 1.609 | 1.968 | 1.857 | 1.460 | 2.827 | 2.797 | 1.396 | 4.955 | 1.720 | 1.042 |
| 5 | 2.519 | 1.200 | 1.287 | 1.380 | 1.208 | 2.245 | 2.185 | 1.231 | 3.703 | 1.351 | 1.191 |
| 2.5 | 2.229 | 1.094 | 1.167 | 1.210 | 0.886 | 1.664 | 1.688 | 1.178 | 2.665 | 1.237 | 1.028 |
| 1.25 | 2.224 | 1.010 | 1.273 | 1.158 | 1.029 | 1.477 | 1.370 | 1.181 | 2.026 | 1.232 | 1.311 |

TABLE 8
Binding affinity results of HFn-Mt-1 2 to Tfr-1

| | | | HFn- | HFn- | HFn- | HFn- | HFn- | HFn- | HFn- | HFn- | HFn- |
|---|---|---|---|---|---|---|---|---|---|---|---|
| | HFn- | HFn- | Mt-12- | Mt-12- | Mt-12- | Mt-12- | Mt-12- | Mt-12- | Mt-12- | Mt-12- | Mt-12- |
| Concentration | WT | Mt-12 | 2-1 | 2-2 | 2-3 | 8-1 | 8-2 | 8-3 | 24-1 | 24-2 | 24-3 |
| 80 | 7.592 | 7.203 | 7.008 | 8.749 | 8.195 | 8.650 | 7.827 | 7.384 | 9.709 | 8.235 | 5.009 |
| 40 | 5.735 | 6.175 | 6.099 | 7.850 | 7.116 | 8.163 | 7.855 | 6.712 | 9.362 | 7.754 | 5.094 |
| 20 | 4.980 | 5.052 | 4.140 | 6.699 | 6.528 | 6.866 | 7.584 | 6.305 | 8.522 | 6.906 | 4.147 |
| 10 | 3.344 | 3.914 | 3.713 | 5.534 | 5.461 | 5.707 | 6.912 | 5.820 | 8.251 | 4.269 | 2.391 |
| 5 | 3.545 | 2.623 | 2.758 | 4.048 | 3.902 | 4.307 | 5.978 | 4.999 | 7.312 | 2.055 | 1.220 |
| 2.5 | 2.771 | 1.518 | 1.752 | 2.729 | 3.441 | 2.683 | 4.370 | 3.733 | 5.204 | 1.292 | 0.977 |
| 1.25 | 2.927 | 1.181 | 1.251 | 2.263 | 2.269 | 1.925 | 2.946 | 2.340 | 4.046 | 1.251 | 0.981 |

TABLE 9
Binding affinity results of HFn-Mt-13 to Tfr-1

| | | | HFn- | HFn- | HFn- | HFn- | HFn- | HFn- | HFn- | HFn- | HFn- |
|---|---|---|---|---|---|---|---|---|---|---|---|
| | HFn- | HFn- | Mt-13- | Mt-13- | Mt-13- | Mt-13- | Mt-13- | Mt-13- | Mt-13- | Mt-13- | Mt-13- |
| Concentration | WT | Mt-13 | 2-1 | 2-2 | 2-3 | 8-1 | 8-2 | 8-3 | 24-1 | 24-2 | 24-3 |
| 80 | 6.147 | 4.850 | 4.754 | 4.198 | 4.023 | 5.119 | 5.364 | 5.051 | 5.176 | 4.230 | 2.529 |
| 40 | 4.306 | 3.976 | 4.540 | 3.762 | 3.501 | 4.550 | 4.322 | 4.659 | 4.732 | 4.056 | 2.259 |
| 20 | 3.943 | 2.984 | 4.112 | 3.108 | 3.154 | 4.214 | 3.930 | 4.116 | 4.438 | 3.682 | 2.143 |
| 10 | 2.495 | 2.511 | 3.151 | 2.729 | 2.515 | 3.910 | 3.736 | 3.676 | 4.090 | 3.201 | 1.865 |
| 5 | 2.863 | 1.773 | 2.632 | 2.119 | 2.097 | 3.295 | 3.170 | 3.012 | 3.515 | 2.299 | 1.550 |
| 2.5 | 1.856 | 1.212 | 1.924 | 1.731 | 1.623 | 2.562 | 2.457 | 2.121 | 2.877 | 1.742 | 1.251 |
| 1.25 | 2.349 | 0.976 | 1.597 | 1.274 | 1.273 | 1.816 | 1.787 | 1.201 | 2.068 | 1.198 | 0.869 |

The affinity of HFn-Mt-1 is reduced compared with that of wild type HFn, however, after PEG modification, the affinity of some samples is improved, whereas the HFn affinity of HFn-Mt-1-24-1 is surprisingly stronger than that of the wild type HFn.

The affinity of HFn-Mt-9 is reduced compared with that of the wild type HFn, after PEG modification, the affinity of all the samples is improved, but is still inferior to that of the wild type HFn.

The affinity of HFn-Mt-12 at the concentration of less than 7.5 μg/ml is reduced compared with that of the wild type HFn, and there is no difference between the affinity of HFn-Mt-12 at the concentration of more than 7.5 μg/ml and the affinity of wild type HFn. After PEG modification, the affinity of most of the modified samples is close to that of the wild type, and the affinity of some samples even at low concentration is higher than that of the wild type (HFn-Mt-12-24-1, HFn-Mt-12-8-2).

The affinity of HFn-Mt-13 is slightly reduced compared with that of wild-type HFn. Furthermore, the affinity of HFn-Mt-13 in the whole concentration range is close to that of wild type, regardless of no modification or PEG modification.

### Example 4 Conjugation of Ferritin H Subunit Mutant to SN-38

Mutants Mut-12 and Mut-12″ were designed to determine the influence of different Cys sites on conjugation. The mutant Mut-12 is different from HFn-Mut-12 that the Cys of the former at sites C102 and C103 is substituted by Ala, and the Cys of the later is substituted by Ser. Therefore, Mut-12 only retains Cys at site C90, and Cys at other two natural sites are mutated to Ala. Mut-12″ serves as control of HFn-Mut-12, only retains Cys at site C102, the Cys at other two natural sites are mutated to amino acids corresponding to L type ferritin, that is, C90 is mutated as Glu, and C130 is mutated as Ala. Mutant design is as shown in Table 10.

TABLE 10
Design scheme of mutants Mut-12 and Mut-12″

| Number of mutants | Mutation site | | | SEQ ID NO |
|---|---|---|---|---|
| Mut-12 | C90 | C102A | C130A | 28 |
| Mut-12″ | C90E | C102 | C130A | 29 |

Preparation and purification of Mut-12 and Mut-12″ mutant proteins are the same as those in Example 1, and their corresponding amino acid sequences are respectively SEQ ID NO: 28 and SEQ ID NO:29, and the nucleotide sequences optimized by corresponding codons are SEQ ID NO:30 and SEQ ID NO:31.

Conjugation experiment of SN-38 is performed on the prepared Mut-12 and Mut-12″ mutant ferritins. The structure of SN-38 with an appropriate linker (Mal-PEG2-VC-PABC-SN-38) for conjugation is as follows:

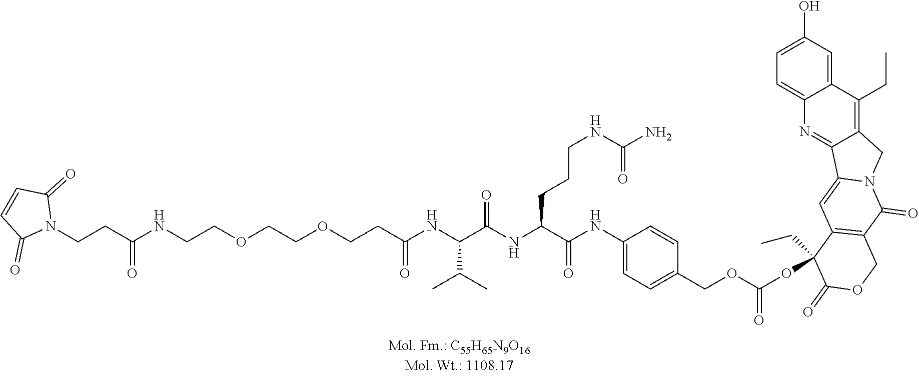 [SMILES: CCc1c2c(nc3ccc(O)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)CCOCCOCCNC(=O)CCN2C(=O)C=CC2=O)C(C)C)cc1]

Mal-PEG2-VC-PABC SN-38 is synthesized by Shanghai Ruizhi Chemistry.

Mut-12 and Mut-12″ were diluted to 1 mg/ml using 50 mM Tris-HCl buffer solution (pH 7.5), Mal-PEG2-SN-38 (dissolved into DMF, a dosing molar ratio: ferritin: SN-38 of 1:8) was added, and the final content of DMF was 10%. The above materials were evenly mixed and underwent standing at room temperature to react. The residue of material Mal-PEG2-VC-PABC SN-38 was detected by RP-HPLC every 30 minutes to monitor the reaction process.

The experimental results show that the reaction activity of Mut-12″ is much lower than that of Mut-12 (as shown in FIG. 4), indicating that cysteine in Loop region is a sulfydryl site that is easier for chemical reaction, and is a preferred selection as a conjugation site.

Sequencing listing
wild type H subunit amino acid sequence
SEQ ID NO: 1
TTASTSQVRQNYHQDSEAAINRQINLELYASYVYLSMSYYFDRDDVALKNFAKYFLHQSH
EEREHAEKLMKLQNQRGGRIFLQDIKKPDCDDWESGLNAMECALHLEKNVNQSLLELH
KLATDKNDPHLCDFIETHYLNEQVKAIKELGDHVTNLRKMGAPESGLAEYLFDKHTLGD
SDNES
HFn-Mut-1
SEQ ID NO: 2
TTASTSQVRQNYHQDSEAAINRQINLELYASYVYLSMSYYFDRDDVALKNFAKYFLHQSH

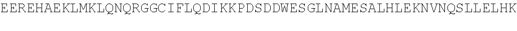

LATDKNDPHLSDFIETHYLNEQVKAIKELGDHVTNLRKMGAPESGLAEYLFDKHTLGDSD
NES
HFn-Mut-2
SEQ ID NO: 3
TTASTSQVRQNYHQDSEAAINRQINLELYASYVYLSMSYYFDRDDVALKNFAKYFLHQSH

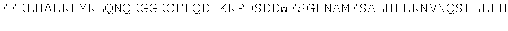

KLATDKNDPHLSDFIETHYLNEQVKAIKELGDHVTNLRKMGAPESGLAEYLFDKHTLGDS
DNES
HFn-Mut-3
SEQ ID NO: 4
TTASTSQVRQNYHQDSEAAINRQINLELYASYVYLSMSYYFDRDDVALKNFAKYFLHQSH

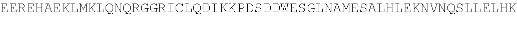

LATDKNDPHLSDFIETHYLNEQVKAIKELGDHVTNLRKMGAPESGLAEYLFDKHTLGDSD
NES
HFn-Mut-4
SEQ ID NO: 5
TTASTSQVRQNYHQDSEAAINRQINLELYASYVYLSMSYYFDRDDVALKNFAKYFLHQSH

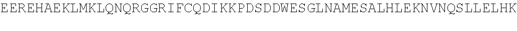

LATDKNDPHLSDFIETHYLNEQVKAIKELGDHVTNLRKMGAPESGLAEYLFDKHTLGDSD
NES
HFn-Mut-5
SEQ ID NO: 6
TTASTSQVRQNYHQDSEAAINRQINLELYASYVYLSMSYYFDRDDVALKNFAKYFLHQSH

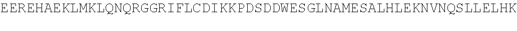

LATDKNDPHLSDFIETHYLNEQVKAIKELGDHVTNLRKMGAPESGLAEYLFDKHTLGDSD
NES
HFn-Mut-6
SEQ ID NO: 7
TTASTSQVRQNYHQDSEAAINRQINLELYASYVYLSMSYYFDRDDVALKNFAKYFLHQSH

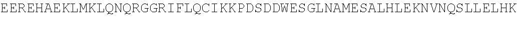

LATDKNDPHLSDFIETHYLNEQVKAIKELGDHVTNLRKMGAPESGLAEYLFDKHTLGDSD
NES
HFn-Mut-7
SEQ ID NO: 8
TTASTSQVRQNYHQDSEAAINRQINLELYASYVYLSMSYYFDRDDVALKNFAKYFLHQSH

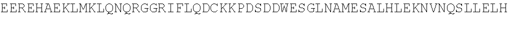

KLATDKNDPHLSDFIETHYLNEQVKAIKELGDHVTNLRKMGAPESGLAEYLFDKHTLGDS
DNES
HFn-Mut-8
SEQ ID NO: 9
TTASTSQVRQNYHQDSEAAINRQINLELYASYVYLSMSYYFDRDDVALKNFAKYFLHQSH

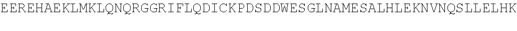

LATDKNDPHLSDFIETHYLNEQVKAIKELGDHVTNLRKMGAPESGLAEYLFDKHTLGDSD
NES
HFn-Mut-9
SEQ ID NO: 10
TTASTSQVRQNYHQDSEAAINRQINLELYASYVYLSMSYYFDRDDVALKNFAKYFLHQSH

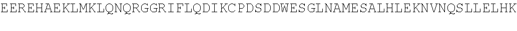

LATDKNDPHLSDFIETHYLNEQVKAIKELGDHVTNLRKMGAPESGLAEYLFDKHTLGDSD
NES
HFn-Mut-10
SEQ ID NO: 11
TTASTSQVRQNYHQDSEAAINRQINLELYASYVYLSMSYYFDRDDVALKNFAKYFLHQSH

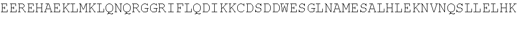

LATDKNDPHLSDFIETHYLNEQVKAIKELGDHVTNLRKMGAPESGLAEYLFDKHTLGDSD
NES
HFn-Mut-11
SEQ ID NO: 12
TTASTSQVRQNYHQDSEAAINRQINLELYASYVYLSMSYYFDRDDVALKNFAKYFLHQSH

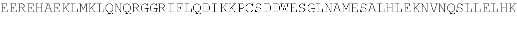

LATDKNDPHLSDFIETHYLNEQVKAIKELGDHVTNLRKMGAPESGLAEYLFDKHTLGDSD
NES
HFn-Mut-12
SEQ ID NO: 13
TTASTSQVRQNYHQDSEAAINRQINLELYASYVYLSMSYYFDRDDVALKNFAKYFLHQSH

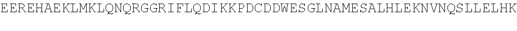

LATDKNDPHLSDFIETHYLNEQVKAIKELGDHVTNLRKMGAPESGLAEYLFDKHTLGDSD
NES
HFn-Mut-13
SEQ ID NO: 14
TTASTSQVRQNYHQDSEAAINRQINLELYASYVYLSMSYYFDRDDVALKNFAKYFLHQSH

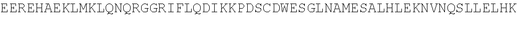

LATDKNDPHLSDFIETHYLNEQVKAIKELGDHVTNLRKMGAPESGLAEYLFDKHTLGDSD
NES
HFn-Mut-1
SEQ ID NO: 15
ACCACCGCAAGTACCTCACAGGTGCGCCAGAATTATCATCAGGATAGCGAAGCAGCCA
TTAATCGTCAGATTAATCTGGAACTGTATGCCTCTTATGTGTATCTGTCTATGAGCTATTA
TTTTGATCGCGATGATGTGGCCCTGAAAAATTTTGCCAAATATTTTCTGCATCAGTCTCA
TGAAGAACGCGAACATGCCGAAAAACTGATGAAACTGCAGAATCAGCGTGGTGGTTG
TATTTTTCTGCAGGATATTAAAAAACCGGATTCAGATGATTGGGAAAGCGGCCTGAATG
CGATGGAAAGCGCCTTACATTTAGAAAAAAATGTTAATCAGTCACTGCTGGAACTGCAT
AAACTGGCAACCGATAAAAATGATCCGCATCTGAGTGATTTTATTGAAACCCATTATCT
GAATGAACAGGTTAAAGCAATTAAAGAATTAGGCGATCATGTGACCAATTTACGTAAAA
TGGGCGCCCCGGAAAGTGGCTTAGCCGAATATCTGTTTGATAAACATACCTTAGGCGAT
AGTGATAATGAATCT
HFn-Mut-2
SEQ ID NO: 16
ACCACCGCAAGTACCTCACAGGTGCGTCAGAATTATCATCAGGATAGCGAAGCAGCAA
TTAATCGCCAGATTAATTTAGAACTGTATGCAAGCTATGTGTATCTGAGTATGAGCTATTA
TTTTGATCGCGATGATGTGGCCCTGAAAAATTTTGCCAAATATTTTCTGCATCAGTCTCA
TGAAGAACGCGAACATGCCGAAAAACTGATGAAATTACAGAATCAGCGTGGTGGTCG
TTGTTTTCTGCAGGATATTAAAAAACCGGATAGCGATGATTGGGAAAGTGGCCTGAATG
CTATGGAAAGTGCCTTACATTTAGAAAAAAATGTTAATCAGTCTCTGCTGGAACTGCAT
AAACTGGCAACCGATAAAAATGATCCGCATCTGTCAGATTTTATTGAAACCCATTATCT
GAATGAACAGGTTAAAGCCATTAAAGAACTGGGTGATCATGTGACCAATTTACGTAAA
ATGGGCGCCCCGGAAAGCGGCTTAGCCGAATATCTGTTTGATAAACATACCTTAGGCGA
TAGTGATAATGAATCT
HFn-Mut-3
SEQ ID NO: 17
ACCACCGCAAGTACCTCACAGGTGCGTCAGAATTATCATCAGGATAGCGAAGCAGCAA
TTAATCGCCAGATTAATTTAGAACTGTATGCAAGCTATGTGTATCTGTCTATGTCTTATTA
TTTTGATCGCGATGATGTTGCCCTGAAAAATTTTGCCAAATATTTTCTGCATCAGTCTCA
TGAAGAACGCGAACATGCCGAAAAACTGATGAAACTGCAGAATCAGCGTGGTGGTCG
TATTTGTTTACAGGATATTAAAAAACCGGATTCAGATGATTGGGAAAGTGGCCTGAATG
CAATGGAAAGTGCCTTACATCTGGAAAAAAATGTTAATCAGAGCCTGCTGGAACTGCA
TAAACTGGCAACCGATAAAAATGATCCGCATCTGAGTGATTTTATTGAAACCCATTATCT
GAATGAACAGGTTAAAGCCATTAAAGAACTGGGCGATCATGTGACCAATTTACGTAAA
ATGGGCGCCCCGGAAAGCGGCTTAGCCGAATATCTGTTTGATAAACATACCTTAGGCGA
TAGTGATAATGAATCT
HFn-Mut-4
SEQ ID NO: 18
ACCACCGCAAGTACCTCACAGGTGCGCCAGAATTATCATCAGGATAGTGAAGCAGCAA
TTAATCGTCAGATTAATCTGGAACTGTATGCAAGCTATGTGTATCTGTCTATGTCTTATTA
TTTTGATCGTGATGATGTGGCCCTGAAAAATTTTGCCAAATATTTTCTGCATCAGTCTCA
TGAAGAACGCGAACATGCCGAAAAACTGATGAAACTGCAGAATCAGCGTGGTGGTCG
CATTTTTTGTCAGGATATTAAAAAACCGGATAGCGATGATTGGGAAAGCGGCCTGAATG
CGATGGAAAGTGCCTTACATTTAGAAAAAAATGTTAATCAGAGCCTGCTGGAACTGCAT
AAACTGGCAACCGATAAAAATGATCCGCATCTGAGCGATTTTATTGAAACCCATTATCT
GAATGAACAGGTTAAAGCCATTAAAGAATTAGGCGATCATGTTACCAATTTACGTAAAA
TGGGCGCCCCGGAAAGTGGCTTAGCCGAATATCTGTTTGATAAACATACCTTAGGCGAT
AGTGATAATGAATCT
HFn-Mut-5
SEQ ID NO: 19
ACCACCGCCTCTACCTCACAGGTGCGCCAGAATTATCATCAGGATAGCGAAGCAGCCA
TTAATCGTCAGATTAATCTGGAACTGTATGCCTCTTATGTGTATCTGAGTATGAGCTATTA
TTTTGATCGTGATGATGTGGCCCTGAAAAATTTTGCCAAATATTTTCTGCATCAGTCTCA
TGAAGAACGCGAACATGCCGAAAAACTGATGAAATTACAGAATCAGCGTGGTGGTCGT
ATTTTTCTGTGTGATATTAAAAAACCGGATTCAGATGATTGGGAAAGCGGCCTGAATGC
GATGGAAAGTGCACTGCATCTGGAAAAAAATGTTAATCAGTCACTGTTAGAACTGCAT
AAACTGGCAACCGATAAAAATGATCCGCATTTAAGCGATTTTATTGAAACCCATTATCT
GAATGAACAGGTTAAAGCAATTAAAGAACTGGGCGATCATGTTACCAATTTACGCAAA
ATGGGCGCCCCGGAAAGTGGCTTAGCCGAATATCTGTTTGATAAACATACCTTAGGCGA
TAGTGATAATGAATCT
HFn-Mut-6
SEQ ID NO: 20
ACCACCGCAAGTACCTCACAGGTGCGTCAGAATTATCATCAGGATAGCGAAGCAGCAA
TTAATCGCCAGATTAATTTAGAACTGTATGCAAGCTATGTGTATCTGTCTATGTCATATTA
TTTTGATCGTGATGATGTTGCCCTGAAAAATTTTGCCAAATATTTTCTGCATCAGTCTCA
TGAAGAACGCGAACATGCCGAAAAACTGATGAAACTGCAGAATCAGCGCGGTGGTCG
CATTTTTCTGCAGTGTATTAAAAAACCGGATAGTGATGATTGGGAAAGCGGCCTGAATG
CGATGGAAAGTGCCTTACATCTGGAAAAAAATGTTAATCAGAGCCTGCTGGAATTACAT
AAACTGGCAACCGATAAAAATGATCCGCATCTGTCAGATTTTATTGAAACCCATTATCT
GAATGAACAGGTTAAAGCCATTAAAGAACTGGGCGATCATGTGACCAATTTACGTAAA
ATGGGCGCCCCGGAAAGTGGCTTAGCCGAATATCTGTTTGATAAACATACCTTAGGCGA
TAGCGATAATGAATCT
HFn-Mut-7
SEQ ID NO: 21
ACCACCGCCTCTACCTCACAGGTGCGTCAGAATTATCATCAGGATAGCGAAGCAGCCAT
TAATCGCCAGATTAATCTGGAACTGTATGCAAGCTATGTGTATCTGTCTATGTCTTATTAT
TTTGATCGTGATGATGTTGCACTGAAAAATTTTGCCAAATATTTTCTGCATCAGTCTCAT
GAAGAACGCGAACATGCCGAAAAACTGATGAAATTACAGAATCAGCGCGGTGGTCGT
ATTTTTCTGCAGGATTGTAAAAAACCGGATAGTGATGATTGGGAAAGTGGCCTGAATGC
AATGGAAAGTGCCCTGCATTTAGAAAAAAATGTTAATCAGAGTTTACTGGAATTACATA
AACTGGCAACCGATAAAAATGATCCGCATCTGAGCGATTTTATTGAAACCCATTATCTG
AATGAACAGGTTAAAGCAATTAAAGAACTGGGCGATCATGTGACCAATTTACGCAAAA
TGGGCGCCCCGGAAAGCGGCTTAGCCGAATATCTGTTTGATAAACATACCTTAGGCGAT
TCAGATAATGAATCT
HFn-Mut-8
SEQ ID NO: 22
ACCACCGCCTCTACCTCACAGGTGCGTCAGAATTATCATCAGGATAGCGAAGCAGCCAT
TAATCGCCAGATTAATTTAGAACTGTATGCAAGCTATGTGTATCTGAGTATGAGCTATTAT
TTTGATCGTGATGATGTTGCCCTGAAAAATTTTGCCAAATATTTTCTGCATCAGTCTCAT
GAAGAACGCGAACATGCCGAAAAACTGATGAAATTACAGAATCAGCGCGGTGGTCGC
ATTTTTCTGCAGGATATTTGTAAACCGGATAGCGATGATTGGGAAAGTGGCCTGAATGC
AATGGAAAGTGCCTTACATCTGGAAAAAAATGTTAATCAGTCACTGCTGGAACTGCAT
AAACTGGCAACCGATAAAAATGATCCGCATCTGTCAGATTTTATTGAAACCCATTATCT
GAATGAACAGGTTAAAGCAATTAAAGAACTGGGCGATCATGTGACCAATTTACGTAAA
ATGGGCGCCCCGGAAAGCGGCTTAGCCGAATATCTGTTTGATAAACATACCTTAGGCGA
TAGTGATAATGAATCT
HFn-Mut-9
SEQ ID NO: 23
ACCACCGCAAGTACCTCACAGGTGCGTCAGAATTATCATCAGGATAGCGAAGCAGCAA
TTAATCGCCAGATTAATTTAGAACTGTATGCCTCTTATGTGTATCTGTCTATGAGCTATTA
TTTTGATCGTGATGATGTTGCCCTGAAAAATTTTGCCAAATATTTTCTGCATCAGTCTCA
TGAAGAACGCGAACATGCCGAAAAACTGATGAAACTGCAGAATCAGCGCGGTGGTCG
CATTTTTCTGCAGGATATTAAATGTCCGGATAGTGATGATTGGGAAAGCGGCCTGAATG
CGATGGAAAGTGCACTGCATCTGGAAAAAAATGTTAATCAGAGCCTGCTGGAATTACA
TAAACTGGCAACCGATAAAAATGATCCGCATCTGTCAGATTTTATTGAAACCCATTATCT
GAATGAACAGGTTAAAGCCATTAAAGAACTGGGCGATCATGTGACCAATTTACGTAAA
ATGGGCGCCCCGGAAAGTGGCTTAGCCGAATATCTGTTTGATAAACATACCTTAGGCGA
TAGCGATAATGAATCT
HFn-Mut-10
SEQ ID NO: 24
ACCACCGCAAGTACCTCACAGGTGCGTCAGAATTATCATCAGGATAGCGAAGCAGCCA
TTAATCGCCAGATTAATCTGGAACTGTATGCCTCTTATGTGTATCTGTCTATGAGCTATTA
TTTTGATCGCGATGATGTTGCCCTGAAAAATTTTGCCAAATATTTTCTGCATCAGTCTCA
TGAAGAACGCGAACATGCCGAAAAACTGATGAAATTACAGAATCAGCGTGGTGGTCGT
ATTTTTCTGCAGGATATTAAAAAATGTGATTCAGATGATTGGGAAAGTGGCCTGAATGC
GATGGAAAGCGCCTTACATTTAGAAAAAAATGTTAATCAGTCACTGCTGGAACTGCATA
AACTGGCAACCGATAAAAATGATCCGCATCTGAGTGATTTTATTGAAACCCATTATCTG
AATGAACAGGTTAAAGCAATTAAAGAACTGGGCGATCATGTGACCAATTTACGTAAAA
TGGGTGCACCGGAAAGCGGCTTAGCCGAATATCTGTTTGATAAACATACCTTAGGCGAT
AGTGATAATGAATCT
HFn-Mut-11
SEQ ID NO: 25
ACCACCGCAAGTACCTCACAGGTGCGTCAGAATTATCATCAGGATAGCGAAGCAGCCA
TTAATCGCCAGATTAATCTGGAACTGTATGCCTCTTATGTGTATCTGTCTATGAGCTATTA
TTTTGATCGCGATGATGTTGCCCTGAAAAATTTTGCCAAATATTTTCTGCATCAGAGTCA
TGAAGAACGTGAACATGCCGAAAAACTGATGAAATTACAGAATCAGCGCGGTGGTCGT
ATTTTTCTGCAGGATATTAAAAAACCGTGTAGCGATGATTGGGAAAGCGGCCTGAATGC
GATGGAAAGTGCACTGCATTTAGAAAAAAATGTTAATCAGTCTCTGCTGGAATTACATA
AACTGGCAACCGATAAAAATGATCCGCATCTGAGCGATTTTATTGAAACCCATTATCTG
AATGAACAGGTTAAAGCAATTAAAGAACTGGGTGATCATGTGACCAATTTACGCAAAA
TGGGCGCCCCGGAAAGTGGCTTAGCCGAATATCTGTTTGATAAACATACCTTAGGCGAT
AGTGATAATGAATCT
HFn-Mut-12
SEQ ID NO: 26
ACCACCGCCTCTACCTCACAGGTTCGTCAGAATTATCATCAGGATAGTGAAGCAGCAAT
TAATCGCCAGATTAATTTAGAACTGTATGCAAGCTATGTGTATCTGAGTATGAGCTATTAT
TTTGATCGCGATGATGTGGCCCTGAAAAATTTTGCCAAATATTTTCTGCATCAGTCTCAT
GAAGAACGCGAACATGCCGAAAAACTGATGAAACTGCAGAATCAGCGTGGTGGTCGC
ATTTTTCTGCAGGATATTAAAAAACCGGATTGTGATGATTGGGAAAGTGGCCTGAATGC
TATGGAAAGTGCCTTACATCTGGAAAAAAATGTTAATCAGTCACTGCTGGAATTACATA
AACTGGCAACCGATAAAAATGATCCGCATCTGTCAGATTTTATTGAAACCCATTATCTG
AATGAACAGGTTAAAGCCATTAAAGAACTGGGTGATCATGTTACCAATTTACGTAAAAT
GGGCGCACCGGAAAGCGGCTTAGCCGAATATCTGTTTGATAAACATACCTTAGGCGATA
GCGATAATGAATCT
HFn-Mut-13
SEQ ID NO: 27
ACCACCGCCTCTACCTCACAGGTGCGCCAGAATTATCATCAGGATAGCGAAGCAGCCA
TTAATCGTCAGATTAATTTAGAACTGTATGCAAGCTATGTGTATCTGAGTATGAGCTATTA
TTTTGATCGCGATGATGTTGCCCTGAAAAATTTTGCCAAATATTTTCTGCATCAGTCTCA
TGAAGAACGCGAACATGCCGAAAAACTGATGAAATTACAGAATCAGCGTGGTGGTCG
CATTTTTCTGCAGGATATTAAAAAACCGGATTCTTGTGATTGGGAAAGCGGCCTGAATG
CAATGGAAAGTGCCTTACATCTGGAAAAAAATGTTAATCAGTCACTGCTGGAACTGCAT
AAACTGGCAACCGATAAAAATGATCCGCATCTGTCAGATTTTATTGAAACCCATTATCT
GAATGAACAGGTTAAAGCAATTAAAGAACTGGGCGATCATGTGACCAATTTACGTAAA
ATGGGCGCCCCGGAAAGTGGCTTAGCCGAATATCTGTTTGATAAACATACCTTAGGCGA
TAGTGATAATGAATCT
Mut-12
SEQ ID NO: 28
TTASTSQVRQNYHQDSEAAINRQINLELYASYVYLSMSYYFDRDDVALKNFAKYFLHQSH

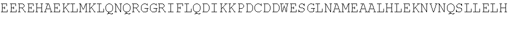

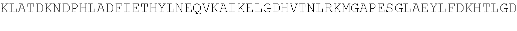

SDNES
Mut-12″
SEQ ID NO: 29
TTASTSQVRQNYHQDSEAAINRQINLELYASYVYLSMSYYFDRDDVALKNFAKYFLHQSH

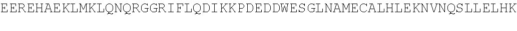

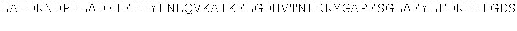

DNES
Mut-12
SEQ ID NO: 30
ACCACCGCAAGTACCTCTCAGGTGCGCCAGAATTATCATCAGGATAGCGAAGCAGCAA
TTAATCGTCAGATTAATCTGGAACTGTATGCAAGCTATGTGTATCTGTCTATGTCTTATTA
TTTTGATCGCGATGATGTGGCACTGAAAAATTTTGCAAAATATTTTCTGCATCAGTCACA
TGAAGAACGCGAACATGCAGAAAAACTGATGAAACTTCAAAATCAGCGTGGTGGTCG
TATTTTTTTGCAAGATATTAAAAAACCGGATTGTGATGATTGGGAAAGTGGCCTGAATG
CAATGGAAGCAGCACTGCATCTGGAAAAAAATGTTAATCAGAGCCTGCTGGAACTGCA
TAAACTGGCAACCGATAAAAATGATCCGCATCTGGCAGATTTTATTGAAACCCATTATCT
GAATGAACAGGTTAAAGCAATTAAAGAACTGGGCGATCATGTTACCAATCTGCGTAAA
ATGGGCGCACCGGAAAGCGGCCTGGCAGAATATCTGTTTGATAAACATACCCTGGGCG
ATAGTGATAATGAAAGC
Mut-12″
SEQ ID NO: 31
ACCACCGCAAGTACCTCTCAGGTGCGCCAGAATTATCATCAGGATAGCGAAGCAGCAA
TTAATCGTCAGATTAATCTGGAACTGTATGCAAGCTATGTGTATCTGTCTATGTCTTATTA
TTTTGATCGCGATGATGTGGCACTGAAAAATTTTGCAAAATATTTTCTGCATCAGTCACA
TGAAGAACGCGAACATGCAGAAAAACTGATGAAACTACAGAATCAGCGTGGTGGTCG
TATTTTTCTCCAGGATATTAAAAAACCGGATGAAGATGATTGGGAAAGTGGCCTGAATG
CAATGGAATGTGCACTGCATCTGGAAAAAAATGTTAATCAGAGCCTGCTGGAACTGCA
TAAACTGGCAACCGATAAAAATGATCCGCATCTGGCAGATTTTATTGAAACCCATTATCT
GAATGAACAGGTTAAAGCAATTAAAGAACTGGGCGATCATGTTACCAATCTGCGTAAA
ATGGGCGCACCGGAAAGCGGCCTGGCAGAATATCTGTTTGATAAACATACCCTGGGCG
ATAGTGATAATGAAAGC
wild type ferritin light chain (L) subunit
SEQ ID NO: 32
SSQIRQNYS TDVEAAVNSL VNLYLQASYT YLSLGFYFDR DDVALEGVSH FFRELAEEKR
EGYERLLKMQ NQRGGRALFQ DIKKPAEDEW GKTPDAMKAA MALEKKLNQA
LLDLHALGSA RTDPHLCDFL ETHFLDEEVK LIKKMGDHLT NLHRLGGPEA
GLGEYLFERL TLKHD

1. A ferritin heavy chain (H) subunit mutant polypeptide, which, as compared to a wild type ferritin H subunit, comprises one cysteine residue in the loop region, the cysteine at a position corresponding to position 102 of SEQ ID NO:1 is substituted, and optionally, the cysteine at a position corresponding to position 130 of SEQ ID NO:1 is substituted.

2. The mutant polypeptide according to claim 1, wherein as compared to a wild type ferritin H subunit, the mutant polypeptide comprises a cysteine at a position corresponding to position 90 of SEQ ID NO:1, and the cysteines at positions corresponding to position 102 and position 130 of SEQ ID NO:1 are substituted in the mutant polypeptide.

3. The mutant polypeptide according to claim 2, wherein in the mutant polypeptide, the cysteines at positions corresponding to position 102 and position 130 of SEQ ID NO:1 are substituted by amino acids selected from serine, threonine, asparagine, glutamine, glutamic acid, aspartic acid, lysine, arginine, histidine, alanine and glycine, preferably serine or amino acids at corresponding positions of a wild type ferritin light chain (L) subunit polypeptide.

4. The mutant polypeptide according to claim 1, wherein as compared to the wild type ferritin H subunit, the cysteines at positions corresponding to position 90 and position 102 of SEQ ID NO:1 are substituted in the mutant polypeptide; and the amino acid at a position corresponding to one of position 79, 80, 81, 82, 83, 84, 85, 86, 87, 88, 89 and 91 of SEQ ID NO:1 is substituted by cysteine in the mutant polypeptide,
optionally, the cysteine at a position corresponding to position 130 of SEQ ID NO:1 is substituted in the mutant polypeptide.

5. The mutant polypeptide according to claim 4, wherein in the mutant polypeptide, the cysteines at positions corresponding to position 90, 102 and/or 103 of SEQ ID NO:1 are substituted by amino acids selected from serine, threonine, asparagine, glutamine, glutamic acid, aspartic acid, lysine, arginine, histidine, alanine and glycine, preferably serine or amino acids at corresponding positions of a wild type ferritin light chain (L) subunit polypeptide.

6. The mutant polypeptide according to claim 4 or 5, wherein, in the mutant polypeptide,
1) the amino acid residue such as arginine residue (R) at a position corresponding to position 79 of SEQ ID NO:1 is substituted by cysteine residue (C);
2) the amino acid residue such as isoleucine residue I at a position corresponding to position 80 of SEQ ID NO:1 is substituted by cysteine residue;
3) the amino acid residue such as phenylalanine residue F at a position corresponding to position 81 of SEQ ID NO:1 is substituted by cysteine residue;
4) the amino acid residue such as leucine residue L at a position corresponding to position 82 of SEQ ID NO:1 is substituted by cysteine residue;
5) the amino acid residue such as glutamine residue Q at a position corresponding to position 83 of SEQ ID NO:1 is substituted by cysteine residue;
6) the amino acid residue such as aspartate residue D at a position corresponding to position 84 of SEQ ID NO:1 is substituted by cysteine residue;
7) the amino acid residue such as isoleucine residue I at a position corresponding to position 85 of SEQ ID NO:1 is substituted by cysteine residue;
8) the amino acid residue such as lysine residue K at a position corresponding to position 86 of SEQ ID NO:1 is substituted by cysteine residue;
9) the amino acid residue such as lysine residue K at a position corresponding to position 87 of SEQ ID NO:1 is substituted by cysteine residue;
10) the amino acid residue such as proline residue P at a position corresponding to position 88 of SEQ ID NO:1 is substituted by cysteine residue;
11) the amino acid residue such as aspartate residue D at a position corresponding to position 89 of SEQ ID NO:1 is substituted by cysteine residue; or
12) the amino acid residue such as aspartate residue D at a position corresponding to position 91 of SEQ ID NO:1 is substituted by cysteine residue;

7. The mutant polypeptide according to claim 1, wherein the mutant polypeptide comprises an amino acid sequence selected from one of SEQ ID NOs:2-14.

8. The mutant polypeptide according to any one of claims 1-7, wherein the mutant polypeptide can be assembled into a cage protein and/or conferring the cage protein with an ability of specifically binding to a TfR1 receptor after being assembled into the cage protein.

9. A polypeptide conjugate, comprising the ferritin H subunit mutant polypeptide according to any one of claims 1-8 and a functional moiety conjugated to the ferritin H subunit mutant polypeptide through the sulfydryl group of the ferritin H subunit mutant polypeptide.

10. The polypeptide conjugate according to claim 9, wherein the functional moiety is selected from a therapeutic molecule, a detectable molecule or a targeting molecule.

11. The polypeptide conjugate according to claim 10, wherein the therapeutic molecule is selected from a small molecule drug, a therapeutic polypeptide and a therapeutic antibody, for example, the therapeutic molecule is SN38.

12. The polypeptide conjugate according to claim 10, wherein the detectable molecule is selected from a fluorescent molecule, a luminous chemical, an enzyme, an isotope and a label.

13. The polypeptide conjugate according to claim 10, wherein the targeting molecule is a targeting antibody.

14. The polypeptide conjugate according to any one of claims 9-13, wherein the functional moiety is conjugated to the ferritin H subunit mutant polypeptide through a linker.

15. The polypeptide conjugate according to any one of claims 9-14, wherein the polypeptide conjugate can be assembled into a cage protein and/or conferring the cage protein with the ability of specifically binding to the TfR1 receptor after being assembled into the cage protein.

16. A cage protein, comprising at least one ferritin H subunit mutant polypeptide of any one of claims 1-8 and/or at least one polypeptide conjugate of any one of claims 9-15.

17. The cage protein according to claim 16, comprising 24 said ferritin H subunit mutant polypeptides and/or polypeptide conjugates.

18. The cage protein according to claim 16, wherein the cage protein is formed by assembling 24 said polypeptide conjugates.

19. The cage protein according to claim 16, the cage protein comprising a plurality of the polypeptide conjugates comprising identical or different functional moieties.

20. A cage protein-API complex, wherein the cage protein-API complex comprises the cage protein of any one of claims 16-19 and an active pharmaceutical ingredient (API) loaded inside the cage protein.

21. A pharmaceutical composition, comprising the ferritin H subunit mutant polypeptide of any one of claims 1-8, the polypeptide conjugate of any one of claims 9-15, the cage protein of any one of claims 16-19 and/or the cage protein-API complex of claim 20, and a pharmaceutically acceptable excipient.

22. Use of the ferritin H subunit mutant polypeptide of any one of claims 1-8, the polypeptide conjugate of any one of claims 9-15, the cage protein of any one of claims 16-19, or the cage protein-API complex of claim 20 and/or the pharmaceutical composition of claim 21 in preparation of a medicine.


SEQUENCE LISTING (32 sequences)

SEQ ID NO: 1 - protein, 182 aa, Homo sapiens
  wild type H subunit amino acid sequence
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMECALHLEKNVNQSLLELHKLATDKNDPHLCDFIETHYLNEQVKAIKELGDHVTNLRKMGAPESGLAEYLFDKHTLGDSDNES

SEQ ID NO: 2 - protein, 182 aa, Artificial Sequence
  HFn-Mut-1
TTASTSQVRQNYHQDSEAAINRQINLELYASYVYLSMSYYFDRDDVALKNFAKYFLHQSHEEREHAEKLMKLQNQRGGCIFLQDIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 3 - protein, 182 aa, Artificial Sequence
  HFn-Mut-2
TTASTSQVRQNYHQDSEAAINRQINLELYASYVYLSMSYYFDRDDVALKNFAKYFLHQSHEEREHAEKLMKLQNQRGGRCFLQDIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 4 - protein, 182 aa, Artificial Sequence
  HFn-Mut-3
TTASTSQVRQNYHQDSEAAINRQINLELYASYVYLSMSYYFDRDDVALKNFAKYFLHQSHEEREHAEKLMKLQNQRGGRICLQDIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 5 - protein, 182 aa, Artificial Sequence
  HFn-Mut-4
TTASTSQVRQNYHQDSEAAINRQINLELYASYVYLSMSYYFDRDDVALKNFAKYFLHQSHEEREHAEKLMKLQNQRGGRIFCQDIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 6 - protein, 182 aa, Artificial Sequence
  HFn-Mut-5
TTASTSQVRQNYHQDSEAAINRQINLELYASYVYLSMSYYFDRDDVALKNFAKYFLHQSHEEREHAEKLMKLQNQRGGRIFLCDIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 7 - protein, 182 aa, Artificial Sequence
  HFn-Mut-6
TTASTSQVRQNYHQDSEAAINRQINLELYASYVYLSMSYYFDRDDVALKNFAKYFLHQSHEEREHAEKLMKLQNQRGGRIFLQCIKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 8 - protein, 182 aa, Artificial Sequence
  HFn-Mut-7
TTASTSQVRQNYHQDSEAAINRQINLELYASYVYLSMSYYFDRDDVALKNFAKYFLHQSHEEREHAEKLMKLQNQRGGRIFLQDCKKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 9 - protein, 182 aa, Artificial Sequence
  HFn-Mut-8
TTASTSQVRQNYHQDSEAAINRQINLELYASYVYLSMSYYFDRDDVALKNFAKYFLHQSHEEREHAEKLMKLQNQRGGRIFLQDICKPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 10 - protein, 182 aa, Artificial Sequence
  HFn-Mut-9
TTASTSQVRQNYHQDSEAAINRQINLELYASYVYLSMSYYFDRDDVALKNFAKYFLHQSHEEREHAEKLMKLQNQRGGRIFLQDIKCPDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 11 - protein, 182 aa, Artificial Sequence
  HFn-Mut-10
TTASTSQVRQNYHQDSEAAINRQINLELYASYVYLSMSYYFDRDDVALKNFAKYFLHQSHEEREHAEKLMKLQNQRGGRIFLQDIKKCDSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 12 - protein, 182 aa, Artificial Sequence
  HFn-Mut-11
TTASTSQVRQNYHQDSEAAINRQINLELYASYVYLSMSYYFDRDDVALKNFAKYFLHQSHEEREHAEKLMKLQNQRGGRIFLQDIKKPCSDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 13 - protein, 182 aa, Artificial Sequence
  HFn-Mut-12
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 14 - protein, 182 aa, Artificial Sequence
  HFn-Mut-13
TTASTSQVRQNYHQDSEAAINRQINLELYASYVYLSMSYYFDRDDVALKNFAKYFLHQSHEEREHAEKLMKLQNQRGGRIFLQDIKKPDSCDWESGLNAMESALHLEKNVNQSLLELHKLATDKNDPHLSDFIETHYLNEQVKAIKELGDHVTNLRKMGAPESGLAEYLFDKHTLGDSDNES

SEQ ID NO: 15 - DNA, 546 bp, Artificial Sequence
  HFn-Mut-1
accaccgcaagtacctcacaggtgcgccagaattatcatcaggatagcgaagcagccattaatcgtcagattaatctggaactgtatgcctcttatgtgtatctgtctatgagctattattttgatcgcgatgatgtggccctgaaaaattttgccaaatattttctgcatcagtctcatgaagaacgcgaacatgccgaaaaactgatgaaactgcagaatcagcgtggtggttgtatttttctgcaggatattaaaaaaccggattcagatgattgggaaagcggcctgaatgcgatggaaagcgccttacatttagaaaaaaatgttaatcagtcactgctggaactgcataaactggcaaccgataaaaatgatccgcatctgagtgattttattgaaacccattatctgaatgaacaggttaaagcaattaaagaattaggcgatcatgtgaccaatttacgtaaaatgggcgccccggaaagtggcttagccgaatatctgtttgataaacataccttaggcgatagtgataatgaatct

SEQ ID NO: 16 - DNA, 546 bp, Artificial Sequence
  HFn-Mut-2
accaccgcaagtacctcacaggtgcgtcagaattatcatcaggatagcgaagcagcaattaatcgccagattaatttagaactgtatgcaagctatgtgtatctgagtatgagctattattttgatcgcgatgatgtggccctgaaaaattttgccaaatattttctgcatcagtctcatgaagaacgcgaacatgccgaaaaactgatgaaattacagaatcagcgtggtggtcgttgttttctgcaggatattaaaaaaccggatagcgatgattgggaaagtggcctgaatgctatggaaagtgccttacatttagaaaaaaatgttaatcagtctctgctggaactgcataaactggcaaccgataaaaatgatccgcatctgtcagattttattgaaacccattatctgaatgaacaggttaaagccattaaagaactgggtgatcatgtgaccaatttacgtaaaatgggcgccccggaaagcggcttagccgaatatctgtttgataaacataccttaggcgatagtgataatgaatct

SEQ ID NO: 17 - DNA, 546 bp, Artificial Sequence
  HFn-Mut-3
accaccgcaagtacctcacaggtgcgtcagaattatcatcaggatagcgaagcagcaattaatcgccagattaatttagaactgtatgcaagctatgtgtatctgtctatgtcttattattttgatcgcgatgatgttgccctgaaaaattttgccaaatattttctgcatcagtctcatgaagaacgcgaacatgccgaaaaactgatgaaactgcagaatcagcgtggtggtcgtatttgtttacaggatattaaaaaaccggattcagatgattgggaaagtggcctgaatgcaatggaaagtgccttacatctggaaaaaaatgttaatcagagcctgctggaactgcataaactggcaaccgataaaaatgatccgcatctgagtgattttattgaaacccattatctgaatgaacaggttaaagccattaaagaactgggcgatcatgtgaccaatttacgtaaaatgggcgccccggaaagcggcttagccgaatatctgtttgataaacataccttaggcgatagtgataatgaatct

SEQ ID NO: 18 - DNA, 546 bp, Artificial Sequence
  HFn-Mut-4
accaccgcaagtacctcacaggtgcgccagaattatcatcaggatagtgaagcagcaattaatcgtcagattaatctggaactgtatgcaagctatgtgtatctgtctatgtcttattattttgatcgtgatgatgtggccctgaaaaattttgccaaatattttctgcatcagtctcatgaagaacgcgaacatgccgaaaaactgatgaaactgcagaatcagcgtggtggtcgcattttttgtcaggatattaaaaaaccggatagcgatgattgggaaagcggcctgaatgcgatggaaagtgccttacatttagaaaaaaatgttaatcagagcctgctggaactgcataaactggcaaccgataaaaatgatccgcatctgagcgattttattgaaacccattatctgaatgaacaggttaaagccattaaagaattaggcgatcatgttaccaatttacgtaaaatgggcgccccggaaagtggcttagccgaatatctgtttgataaacataccttaggcgatagtgataatgaatct

SEQ ID NO: 19 - DNA, 546 bp, Artificial Sequence
  HFn-Mut-5
accaccgcctctacctcacaggtgcgccagaattatcatcaggatagcgaagcagccattaatcgtcagattaatctggaactgtatgcctcttatgtgtatctgagtatgagctattattttgatcgtgatgatgtggccctgaaaaattttgccaaatattttctgcatcagtctcatgaagaacgcgaacatgccgaaaaactgatgaaattacagaatcagcgtggtggtcgtatttttctgtgtgatattaaaaaaccggattcagatgattgggaaagcggcctgaatgcgatggaaagtgcactgcatctggaaaaaaatgttaatcagtcactgttagaactgcataaactggcaaccgataaaaatgatccgcatttaagcgattttattgaaacccattatctgaatgaacaggttaaagcaattaaagaactgggcgatcatgttaccaatttacgcaaaatgggcgccccggaaagtggcttagccgaatatctgtttgataaacataccttaggcgatagtgataatgaatct

SEQ ID NO: 20 - DNA, 546 bp, Artificial Sequence
  HFn-Mut-6
accaccgcaagtacctcacaggtgcgtcagaattatcatcaggatagcgaagcagcaattaatcgccagattaatttagaactgtatgcaagctatgtgtatctgtctatgtcatattattttgatcgtgatgatgttgccctgaaaaattttgccaaatattttctgcatcagtctcatgaagaacgcgaacatgccgaaaaactgatgaaactgcagaatcagcgcggtggtcgcatttttctgcagtgtattaaaaaaccggatagtgatgattgggaaagcggcctgaatgcgatggaaagtgccttacatctggaaaaaaatgttaatcagagcctgctggaattacataaactggcaaccgataaaaatgatccgcatctgtcagattttattgaaacccattatctgaatgaacaggttaaagccattaaagaactgggcgatcatgtgaccaatttacgtaaaatgggcgccccggaaagtggcttagccgaatatctgtttgataaacataccttaggcgatagcgataatgaatct

SEQ ID NO: 21 - DNA, 546 bp, Artificial Sequence
  HFn-Mut-7
accaccgcctctacctcacaggtgcgtcagaattatcatcaggatagcgaagcagccattaatcgccagattaatctggaactgtatgcaagctatgtgtatctgtctatgtcttattattttgatcgtgatgatgttgcactgaaaaattttgccaaatattttctgcatcagtctcatgaagaacgcgaacatgccgaaaaactgatgaaattacagaatcagcgcggtggtcgtatttttctgcaggattgtaaaaaaccggatagtgatgattgggaaagtggcctgaatgcaatggaaagtgccctgcatttagaaaaaaatgttaatcagagtttactggaattacataaactggcaaccgataaaaatgatccgcatctgagcgattttattgaaacccattatctgaatgaacaggttaaagcaattaaagaactgggcgatcatgtgaccaatttacgcaaaatgggcgccccggaaagcggcttagccgaatatctgtttgataaacataccttaggcgattcagataatgaatct

SEQ ID NO: 22 - DNA, 546 bp, Artificial Sequence
  HFn-Mut-8
accaccgcctctacctcacaggtgcgtcagaattatcatcaggatagcgaagcagccattaatcgccagattaatttagaactgtatgcaagctatgtgtatctgagtatgagctattattttgatcgtgatgatgttgccctgaaaaattttgccaaatattttctgcatcagtctcatgaagaacgcgaacatgccgaaaaactgatgaaattacagaatcagcgcggtggtcgcatttttctgcaggatatttgtaaaccggatagcgatgattgggaaagtggcctgaatgcaatggaaagtgccttacatctggaaaaaaatgttaatcagtcactgctggaactgcataaactggcaaccgataaaaatgatccgcatctgtcagattttattgaaacccattatctgaatgaacaggttaaagcaattaaagaactgggcgatcatgtgaccaatttacgtaaaatgggcgccccggaaagcggcttagccgaatatctgtttgataaacataccttaggcgatagtgataatgaatct

SEQ ID NO: 23 - DNA, 546 bp, Artificial Sequence
  HFn-Mut-9
accaccgcaagtacctcacaggtgcgtcagaattatcatcaggatagcgaagcagcaattaatcgccagattaatttagaactgtatgcctcttatgtgtatctgtctatgagctattattttgatcgtgatgatgttgccctgaaaaattttgccaaatattttctgcatcagtctcatgaagaacgcgaacatgccgaaaaactgatgaaactgcagaatcagcgcggtggtcgcatttttctgcaggatattaaatgtccggatagtgatgattgggaaagcggcctgaatgcgatggaaagtgcactgcatctggaaaaaaatgttaatcagagcctgctggaattacataaactggcaaccgataaaaatgatccgcatctgtcagattttattgaaacccattatctgaatgaacaggttaaagccattaaagaactgggcgatcatgtgaccaatttacgtaaaatgggcgccccggaaagtggcttagccgaatatctgtttgataaacataccttaggcgatagcgataatgaatct

SEQ ID NO: 24 - DNA, 546 bp, Artificial Sequence
  HFn-Mut-10
accaccgcaagtacctcacaggtgcgtcagaattatcatcaggatagcgaagcagccattaatcgccagattaatctggaactgtatgcctcttatgtgtatctgtctatgagctattattttgatcgcgatgatgttgccctgaaaaattttgccaaatattttctgcatcagtctcatgaagaacgcgaacatgccgaaaaactgatgaaattacagaatcagcgtggtggtcgtatttttctgcaggatattaaaaaatgtgattcagatgattgggaaagtggcctgaatgcgatggaaagcgccttacatttagaaaaaaatgttaatcagtcactgctggaactgcataaactggcaaccgataaaaatgatccgcatctgagtgattttattgaaacccattatctgaatgaacaggttaaagcaattaaagaactgggcgatcatgtgaccaatttacgtaaaatgggtgcaccggaaagcggcttagccgaatatctgtttgataaacataccttaggcgatagtgataatgaatct

SEQ ID NO: 25 - DNA, 546 bp, Artificial Sequence
  HFn-Mut-11
accaccgcaagtacctcacaggtgcgtcagaattatcatcaggatagcgaagcagccattaatcgccagattaatctggaactgtatgcctcttatgtgtatctgtctatgagctattattttgatcgcgatgatgttgccctgaaaaattttgccaaatattttctgcatcagagtcatgaagaacgtgaacatgccgaaaaactgatgaaattacagaatcagcgcggtggtcgtatttttctgcaggatattaaaaaaccgtgtagcgatgattgggaaagcggcctgaatgcgatggaaagtgcactgcatttagaaaaaaatgttaatcagtctctgctggaattacataaactggcaaccgataaaaatgatccgcatctgagcgattttattgaaacccattatctgaatgaacaggttaaagcaattaaagaactgggtgatcatgtgaccaatttacgcaaaatgggcgccccggaaagtggcttagccgaatatctgtttgataaacataccttaggcgatagtgataatgaatct

SEQ ID NO: 26 - DNA, 546 bp, Artificial Sequence
  HFn-Mut-12
accaccgcctctacctcacaggttcgtcagaattatcatcaggatagtgaagcagcaattaatcgccagattaatttagaactgtatgcaagctatgtgtatctgagtatgagctattattttgatcgcgatgatgtggccctgaaaaattttgccaaatattttctgcatcagtctcatgaagaacgcgaacatgccgaaaaactgatgaaactgcagaatcagcgtggtggtcgcatttttctgcaggatattaaaaaaccggattgtgatgattgggaaagtggcctgaatgctatggaaagtgccttacatctggaaaaaaatgttaatcagtcactgctggaattacataaactggcaaccgataaaaatgatccgcatctgtcagattttattgaaacccattatctgaatgaacaggttaaagccattaaagaactgggtgatcatgttaccaatttacgtaaaatgggcgcaccggaaagcggcttagccgaatatctgtttgataaacataccttaggcgatagcgataatgaatct

SEQ ID NO: 27 - DNA, 546 bp, Artificial Sequence
  HFn-Mut-13
accaccgcctctacctcacaggtgcgccagaattatcatcaggatagcgaagcagccattaatcgtcagattaatttagaactgtatgcaagctatgtgtatctgagtatgagctattattttgatcgcgatgatgttgccctgaaaaattttgccaaatattttctgcatcagtctcatgaagaacgcgaacatgccgaaaaactgatgaaattacagaatcagcgtggtggtcgcatttttctgcaggatattaaaaaaccggattcttgtgattgggaaagcggcctgaatgcaatggaaagtgccttacatctggaaaaaaatgttaatcagtcactgctggaactgcataaactggcaaccgataaaaatgatccgcatctgtcagattttattgaaacccattatctgaatgaacaggttaaagcaattaaagaactgggcgatcatgtgaccaatttacgtaaaatgggcgccccggaaagtggcttagccgaatatctgtttgataaacataccttaggcgatagtgataatgaatct

SEQ ID NO: 28 - protein, 182 aa, Artificial Sequence
  Mut-12
TTASTSQVRQNYHQDSEAAINRQINLELYASYVYLSMSYYFDRDDVALKNFAKYFLHQSHEEREHAEKLMKLQNQRGGRIFLQDIKKPDCDDWESGLNAMEAALHLEKNVNQSLLELHKLATDKNDPHLADFIETHYLNEQVKAIKELGDHVTNLRKMGAPESGLAEYLFDKHTLGDSDNES

SEQ ID NO: 29 - protein, 182 aa, Artificial Sequence
  Mut-12“
TTASTSQVRQNYHQDSEAAINRQINLELYASYVYLSMSYYFDRDDVALKNFAKYFLHQSHEEREHAEKLMKLQNQRGGRIFLQDIKKPDEDDWESGLNAMECALHLEKNVNQSLLELHKLATDKNDPHLADFIETHYLNEQVKAIKELGDHVTNLRKMGAPESGLAEYLFDKHTLGDSDNES

SEQ ID NO: 30 - DNA, 546 bp, Artificial Sequence
  Mut-12
accaccgcaagtacctctcaggtgcgccagaattatcatcaggatagcgaagcagcaattaatcgtcagattaatctggaactgtatgcaagctatgtgtatctgtctatgtcttattattttgatcgcgatgatgtggcactgaaaaattttgcaaaatattttctgcatcagtcacatgaagaacgcgaacatgcagaaaaactgatgaaacttcaaaatcagcgtggtggtcgtatttttttgcaagatattaaaaaaccggattgtgatgattgggaaagtggcctgaatgcaatggaagcagcactgcatctggaaaaaaatgttaatcagagcctgctggaactgcataaactggcaaccgataaaaatgatccgcatctggcagattttattgaaacccattatctgaatgaacaggttaaagcaattaaagaactgggcgatcatgttaccaatctgcgtaaaatgggcgcaccggaaagcggcctggcagaatatctgtttgataaacataccctgggcgatagtgataatgaaagc

SEQ ID NO: 31 - DNA, 546 bp, Artificial Sequence
  Mut-12”
accaccgcaagtacctctcaggtgcgccagaattatcatcaggatagcgaagcagcaattaatcgtcagattaatctggaactgtatgcaagctatgtgtatctgtctatgtcttattattttgatcgcgatgatgtggcactgaaaaattttgcaaaatattttctgcatcagtcacatgaagaacgcgaacatgcagaaaaactgatgaaactacagaatcagcgtggtggtcgtatttttctccaggatattaaaaaaccggatgaagatgattgggaaagtggcctgaatgcaatggaatgtgcactgcatctggaaaaaaatgttaatcagagcctgctggaactgcataaactggcaaccgataaaaatgatccgcatctggcagattttattgaaacccattatctgaatgaacaggttaaagcaattaaagaactgggcgatcatgttaccaatctgcgtaaaatgggcgcaccggaaagcggcctggcagaatatctgtttgataaacataccctgggcgatagtgataatgaaagc

SEQ ID NO: 32 - protein, 174 aa, Homo sapiens
  wild type ferritin light chain (L) subunit
SSQIRQNYSTDVEAAVNSLVNLYLQASYTYLSLGFYFDRDDVALEGVSHFFRELAEEKREGYERLLKMQNQRGGRALFQDIKKPAEDEWGKTPDAMKAAMALEKKLNQALLDLHALGSARTDPHLCDFLETHFLDEEVKLIKKMGDHLTNLHRLGGPEAGLGEYLFERLTLKHD